(19) United States
(12) Patent Application Publication
(10) Pub. No.: US 2025/0090830 A1
(43) Pub. Date: Mar. 20, 2025

(54) Percutaneous Endovascular Centrifugal Heart Pump and Method

(71) Applicant: Cardioforma LLC, North Bay Village, FL (US)

(72) Inventor: David Esteban Paniagua Gonzalez, North Bay Village, FL (US)

(73) Assignee: Cardioforma LLC, North Bay Village, FL (US)

(21) Appl. No.: 18/289,885

(22) PCT Filed: Apr. 24, 2023
(86) PCT No.: PCT/US23/19544

§ 371 Date: Nov. 7, 2023

Related U.S. Application Data
(60) Provisional application No. 63/345,374, filed on May 24, 2022.

(51) Int. Cl. A61M 60/13; A61M 60/237; A61M 60/422; A61M 60/806

(52) U.S. Cl. CPC A61M 60/13; A61M 60/237; A61M 60/422; A61M 60/806

(57) ABSTRACT

Percutaneous heart pump that has centrifugal flow and valve conduit allowing flow in one direction. The present invention is a miniaturized percutaneous endovascular centrifugal pump that incorporates an expandable uniflow valve conduit with valves, a centrifugal impeller, a shaft, a guidewire, a deliverable sheath and extracorporeal couplings to an infusion pump and motor.

This application claims the benefit of U.S. provisional application 63/345,374, filed May 24, 2022, and entitled Percutaneous Endovascular Centrifugal Heart Pump, which application is hereby incorporated by reference in its entirety and made a part of this Application, and also claims the benefit of PCT Application PCT/US23/19544, filed Apr. 24, 2023, and entitled Percutaneous Endovascular Centrifugal Heart Pump and Method, which application is hereby incorporated by reference in its entirety and made a part of this Application

### FIELD OF THE INVENTION

The present invention relates to a percutaneous endovascular centrifugal heart pump to support the failing heart as a bridge to recovery or during high-risk cardiac interventions.

### BACKGROUND OF THE INVENTION

Pumps have been around for centuries. The first rotary pump dates back to Archimedes' screw pump (250 B.C.), used to displace fluid from a lower to a higher plane. The first centrifugal pump was introduced for mud lifting in 1475 in a treatise by Francesco di Giorgio Martini. The physics of pump flow and mathematical interpretation were explained by Daniel Bernoulli and Leonhard Euler in the 1700s who derived the velocity triangles used still today to calculate pump flow.

Pumps are classified as displacement and rotary pumps. Displacement pumps produce intermittent flow with periodic energy transfer; rotary pumps generate continuous flow with energy transfer due to impeller velocity. There are three classical rotary pumps: centrifugal, axial, and mixed flow. Axial pumps use a propeller to advance the fluid's mass on the same axis as the initial flow. Centrifugal pumps generate flow by applying the angular momentum principle to the fluid's mass through the impeller passages advancing the mass of fluid radially. A mixed flow pump uses a combination of centrifugal and axial.

In medicine, displacement pumps have applications for hemodialysis and heart and lung machines. The Jarvik and HeartMate II left ventricular assist devices (LVAD) use axial pumps; the HeartMate III and HeartWare (LVADs) use centrifugal pumps. Dr. Richard Wampler developed the HemoPump (1985), the first percutaneous axial flow pump for supporting the human heart inspired by the Archimedes screw pump. This work was advanced through individuals such as Dr. Helmut Reul and Dr. O. H. Frazier, which led to the development of the Impella device by Thorsten Sieb.

It took almost 60 years of work in the medical field to learn and accept that the human body can function without a pulse. Despite all these efforts, there is still a lot more progress to be made and innovate in this field.

There is a societal need for a low-profile or miniature percutaneous mechanical circulatory support (mPMCS) to treat patients with small or diseased femoral arteries. The main problems with prior art PMCS are sheath size and the impeller rotational speed necessary to generate adequate flow. The AbioMed Impella™ has an outer diameter of 18F (6 mm), increasing the difficulty of accessing the femoral artery. This large catheter is problematic in patients with small access points, tortuous or calcified vessels increasing the risk of complications such as bleeding, tears, dissection, total occlusion, transections, spasms, or embolic events.

The sheath size required to introduce the PMCS is a limiting factor for vessel access and pump performance. The current smallest available device has a sheath size of 6 mm (O.D.) diameter for 2.5 Impella™ (2.5 L/min) and the CP Impella™ (3 L/min) device and for 5.0 Impella™ device (5 L/min), it is recommended the 10 mm diameter HemoShield for vascular access. This sheath size creates a problem because the average common femoral artery diameter is 6.6 mm (3.9 to 8.9 mm). The Impella™ device has a sheath with approximately the same diameter size as the access vessel. Additionally, the 5.0 Impella™, due to its profile, percutaneous insertion is rarely done. The introduction of these large cannulas may jeopardize blood flow, causing lactic acidosis, limb ischemia, and amputations.

Furthermore, large sheaths have flexion difficulties conforming to the human anatomy, especially in tortuous arteries, increasing the stress applied at the vessel's arterial walls, thus leading to complications. For example, the friction produced by the large sheath can dislodge calcium in the artery and the aorta, which can embolize to the heart, limb, kidney, or the brain causing a heart attack, limb ischemia, renal infarct, or stroke in the patient, respectively. Thus, not all patients are candidates for the smallest available device due to the anatomical reasons explained above. Another concern that is a limiting factor is the vessel tortuosity (FIG. 1). This tortuosity is a risk factor for vessel damage when advancing a large sheath. The presence of heavily calcified vessels may cause calcium embolization when advancing the large device. An additional concern is the curvature of the aortic arch and the difficulty of advancing a large sheath catheter without interacting with the aortic wall (FIG. 3).

The present invention satisfies these needs by providing the first miniaturized endovascular percutaneous centrifugal pump in the medical field.

### SUMMARY OF THE INVENTION

The present invention comprises a small insertion profile, housing the valve conduit, shaft, impeller, stator, and guidewire, all inside an 8F-12F (French) sheath. The present invention expands to 10-20 mm during operation, which allows lower impeller speeds of 4,000 to 25,000 RPMs-3 to 6 times lower rotational speeds than current ventricular assist devices (33,000-57,000 RPM). Furthermore, the present invention provides low blood velocities (0.54 m/s) while still generating up to 5 L/min across the uniflow valve conduit; 12 times lower velocities than current technologies (6.25 m/s). This helps minimize blood cell trauma.

The present invention will be the first percutaneous endovascular centrifugal heart pump in the market, providing cardiologists a low-profile device that would facilitate insertion, maneuverability in the human-body minimizing damage to the vessels and complications. Patients who previously were not considered candidates due to vessel size would have access to this therapy.

The present invention fills the unmet need offering a true-low profile device by providing access to patients who currently do not have an option due to their small arteries. The present invention will be 36% smaller in diameter, and it will pump up to 5 L/min at lower rotational velocities.

The reduction in impeller speed and increased valve conduit diameter will lower blood cell damage and facilitate the insertion and advancement of the present invention in patients with tortuous and small vessels who do not qualify for heart support with available technology. The present invention will support the heart to restore cardiac function, giving the patient's heart time to recover. Thus, decreasing the progression to an end-stage heart disease with a medical and economical direct impact.

### BRIEF DESCRIPTION OF THE DRAWINGS

FIG. 1 is an overview of the present invention showing a magnified view; a segmented view removing the sheath; and a segmented view removing the sheath, valve conduit, and frame.

FIGS. 2a and 2b are front and top views, respectively, of the impeller in the closed state with a plurality of levels cut from a single piece of material.

FIGS. 2c and 2d are front and top views, respectively, of the impeller in the open state with a plurality of levels cut from a single piece of material.

FIG. 2e illustrates the top view of the impeller and the vane angle distribution, a.

FIGS. 2f and 2g are perspective views of the impeller.

FIGS. 3a and 3b are side views of the frame of the valve conduit in a closed and expanded state.

FIGS. 3c and 3d are side views of the frame of the valve conduit in the closed and expanded state with a slit on either end.

FIG. 4a is a side view of the arterial version of the present invention illustrating the shaft, frame, impeller, shaft stabilizer, and insertion tip.

FIG. 4b is similar to FIG. 4a but without the impeller.

FIG. 5a illustrates the interaction of the shaft, impeller, shaft stabilizer, and insertion tip.

FIG. 5b is similar to FIG. 5a but illustrates the shaft, impeller, shaft stabilizer, and insertion tip as segmented components.

FIG. 6 is an isometric view of the interaction of the shaft, impeller, shaft stabilizer, and insertion tip.

FIG. 7a is a side view the present invention illustrating the stator, shaft, impeller, valve conduit, frame, and insertion tip.

FIG. 7b is a cross-sectional view of the present invention taken along line A-A of FIG. 7a.

FIG. 8a is a cross-sectional side view of the present invention.

FIG. 8b is a magnified view of the end of the present invention as shown in FIG. 8a showing the shaft, impeller, valve conduit, frame, and insertion tip.

FIG. 9a is a left side view of the present invention illustrating the valve conduit, the valve conduit valves, frame, stator, shaft stabilizer, and insertion tip.

FIG. 9b is a frontal view of the present invention illustrating the valve conduit, the valve conduit valves, frame, stator, shaft stabilizer, and insertion tip.

FIG. 9c is a right side view of the present invention illustrating the valve conduit, the valve conduit valves, frame, stator, shaft stabilizer, and insertion tip.

FIG. 10a is a left side view of the present invention illustrating the valve conduit, the valve conduit valves with a larger opening angle in the open position, frame, stator, shaft stabilizer, sheath, guidewire, and insertion tip.

FIG. 10b is a frontal view of the present invention as seen in FIG. 10a illustrating the valve conduit, the valve conduit valves with a larger opening angle in the open position, frame, stator, shaft stabilizer, sheath, guidewire, and insertion tip.

FIG. 11a is a front view of the arterial version of the present invention illustrating the valve conduit, the valve conduit valves in the closed position, frame, stator, shaft stabilizer, sheath, guidewire, and insertion tip.

FIG. 11b is a side view of the arterial version of the present invention as seen in FIG. 11a with the valve conduit valves in the open position.

FIG. 12a is an isometric view of the arterial version of the present invention illustrating the valves in an open position.

FIG. 12b is an isometric view of the arterial version of the present invention illustrating the valves in a closed position.

FIG. 12c is an isometric view of the arterial version of the present invention illustrating the location of inflow and outflow.

FIG. 13 is a bottom view of the arterial version of the present invention illustrating the impeller, valve conduit, and the valve conduit valves in the open position.

FIG. 14 is a top view of the arterial version of the present invention illustrating the valve conduit, the valve conduit valves in the open position with valve conduit circulating jets.

FIG. 15 is a side view of the venous version of the present invention.

FIG. 16 is a cross-sectional side view of the venous version of the present invention.

FIG. 17 is a magnified cross-sectional side view of the venous version of the present invention's insertion tip and venous shaft stabilizer.

FIG. 18 is a top view of the venous version of the present invention as seen in FIG. 15 illustrating the valve conduit, the valve conduit valves open, and valve conduit circulating flow jets.

FIG. 19a is a side view of the venous version of the present invention illustrating the stator, shaft, frame, venous shaft stabilizer, impeller, and insertion tip.

FIG. 19b is similar to FIG. 19a but without the impeller.

FIG. 20a is a side view of the venous version of the present invention showing the shaft, impeller, venous stabilizer, and insertion tip.

FIG. 20b is similar to FIG. 20a but in a segmented view of the components.

FIG. 21 is an isometric view of the venous version of the present invention showing the shaft, impeller, venous stabilizer, and insertion tip.

FIG. 22 is an isometric, segmented view of the venous version of the present invention showing the shaft, impeller, venous stabilizer, and insertion tip.

FIG. 23 is an isometric view of the venous version of the present invention illustrating the stator, shaft, frame, venous shaft stabilizer, and insertion tip but without the valve conduit.

FIG. 24 is a comparison between the arterial and venous versions of the present invention.

FIG. 25a is a side view of the motor to device connection of the present invention.

FIG. 25b is a cross-sectional of the side view shown in FIG. 25a.

FIG. 26a is another cross-sectional side view of the motor to device connection of the present invention.

FIG. 26b is a detailed, magnified view of the circled area of FIG. 26a.

FIG. 27a is a side view of the motor to device connections of the present invention illustrating the segmented components.

FIG. 27b is a cross-sectional view of the side view shown in FIG. 27a.

FIG. 28 is an isometric view of the motor to device connection shown in FIG. 27a.

FIG. 29a to FIG. 29h illustrate the components that connect the heart pump to the motor of the present invention.

FIG. 30a and FIG. 30b illustrate the unsheathing process of the present invention using a pulling mechanism.

FIG. 31a and FIG. 31b illustrate the unsheathing process of the present invention using a pushing mechanism.

FIG. 32 is a side view of the present invention and its motor.

FIG. 33 illustrates steps 1 and 2 for the insertion of the arterial version of the present invention into a body.

FIG. 34 illustrates steps 3 and 4 for the insertion of the arterial version of the present invention into a body.

FIG. 35 illustrates step 5 for the insertion of the arterial version of the present invention into a body.

FIG. 36 is a cross-sectional view of the heart from step 1 above illustrating the placement of the arterial version of the present invention into a body in the left side of the heart. A wire is located in the left ventricle.

FIG. 37 is a cross-sectional view of the heart from step 2 above illustrating the placement of the arterial version of the present inventions in the left side of the heart and advancing across the aortic valve into the left ventricle.

FIG. 38 is a cross-sectional view of the heart from step 3 above illustrating the placement of the arterial version of the present invention in the left side of the heart and the unsheathing of the present invention across the aortic valve.

FIG. 39 is a cross-sectional view of the heart from step 4 above illustrating the placement of the arterial version of the present invention in the left side of the heart when the present invention is fully unsheathed, and the valve conduit valves are closed.

FIG. 40 is a cross-sectional view of the heart from step 5 above illustrating the placement of the arterial version of the present invention in the left side of the heart when the valves of the valve conduit are fully opened.

FIG. 41a and FIG. 41b illustrate access points by the guide wire 500 for the venous version of the present invention through the femoral vein and jugular vein.

FIG. 42a and FIG. 42b illustrate access points by the guide wire 500 for venous version of the present invention through the subclavian vein and antecubital veins, such as the basilic and cephalic veins.

FIG. 43 illustrates the insertion of the venous version of the present invention into the venous system using the femoral vein.

FIG. 44 is a cross-sectional view of the heart illustrating a method 1 for the placement of the venous version of the present invention in the pulmonary position in the right side of the heart.

FIG. 45 is a cross-sectional view of the heart illustrating a method 2 for the placement of the venous version of the present invention in the pulmonary position in the right side of the heart.

FIG. 46 is a cross-sectional view of the heart illustrating a method 1 for the placement of the venous version of the present invention in the tricuspid position on the right side of the heart introduced via the jugular vein.

FIG. 47 is a cross-sectional view of the heart illustrating a method 2 of the placement of the venous version of the present invention in the tricuspid position on the right side of the heart introduced via the femoral vein.

FIG. 48 is a cross-sectional view of the heart illustrating the placement of the guide wire 500 across the mitral valve into the left ventricle for subsequent placement of the venous version of the present invention. Guide wire 500 enters the heart from the inferior vena cava into the right atrium, crosses the atrial septum into the left atrium, crosses the mitral valve and is positioned into the left ventricle.

FIG. 49 is a cross-sectional view of the heart illustrating the placement of the venous version of the present invention in the mitral valve. The venous version of the present invention enters the heart from the inferior vena cava into the right atrium, crosses the atrial septum into the left atrium, crosses the mitral valve and is positioned into the left ventricle.

FIG. 50 is a cross-sectional view of the heart illustrating the placement of the guide wire 500 across the mitral valve into the left ventricle for placement of the venous version of the present invention. Guide wire 500 enters the heart from the superior vena cava into the right atrium, crosses the atrial septum into the left atrium, crosses the mitral valve and is positioned into the left ventricle.

FIG. 51 is a cross-sectional view of the heart illustrating the placement of the venous version of the present invention in the mitral valve. The venous version of the present invention enters the heart from the superior vena cava into the right atrium, crosses the atrial septum into the left atrium, crosses the mitral valve and is positioned into the left ventricle.

FIG. 52a and FIG. 52b illustrate the flow profile of the present invention with limited opening of the valve conduit valves.

FIG. 53a and FIG. 53b illustrate the flow profile of the present invention with full opening of the valve conduit valves.

FIG. 54a and FIG. 54b are a comparison between a prior art axial impeller and the impeller of the present invention.

FIG. 54c and FIG. 54d are a comparison between the axial flow of a prior art axial impeller and centrifugal flow of the impeller of the present invention.

FIG. 55a and FIG. 55b are side views of the arterial version of the present invention illustrating the placement of microelectromechanical systems (MEMS) pressure sensors 338a/338b.

FIG. 56 is a side view of the venous version of the present inventions illustrating the placement of microelectromechanical systems (MEMS) pressure sensors 338c/338d.

FIG. 57 is a front view of the arterial version of the present invention illustrating the electrical connections and communication between the arterial version of the present invention and its power supply and monitor.

FIG. 58 is another front view of the arterial version of the present invention further illustrating the electrical connections and communication between the arterial version of the present invention and its power supply and monitor.

### DETAILED DESCRIPTION OF THE PREFERRED EMBODIMENT

Characteristics and advantages of the present disclosure and additional features and benefits will be readily apparent to those skilled in the art upon consideration of the following detailed description of exemplary embodiments of the present disclosure and referring to the accompanying figures. It should be understood that the description herein and appended drawings, being of example embodiments, are not intended to limit the claims of this patent or any patent or patent application claiming priority hereto. On the contrary, the intention is to cover all modifications, equivalents and alternatives falling within the spirit and scope of the claims. Changes may be made to the particular embodiments and details disclosed herein without departing from such spirit and scope.

In showing and describing preferred embodiments in the appended figures, common or similar elements are referenced with like or identical reference numerals or are apparent from the figures and/or the description herein. The figures are not necessarily to scale and certain features and certain views of the figures may be shown exaggerated in scale or in schematic in the interest of clarity and conciseness.

As used herein and throughout various portions (and headings) of this patent application, the terms “disclosure”, “present disclosure” and variations thereof are not intended to mean every possible embodiment encompassed by this disclosure or any particular claim(s). Thus, the subject matter of each such reference should not be considered as necessary for, or part of, every embodiment hereof or of any particular claim(s) merely because of such reference.

The term “coupled” and the like, and variations thereof, as used herein and in the appended claims are intended to mean either an indirect or direct connection or engagement. Thus, if a first device couples to a second device, that connection may be through a direct connection, or through an indirect connection via other devices and connections.

The use of the terms “a” and “an” and “the” and similar referents in the context of describing the invention (especially in the context of the following claims) are to be construed to cover both the singular and the plural, unless otherwise indicated herein or clearly contradicted by context. Further, it should be noted that the terms “first,” “second,” and the like herein do not denote any order, quantity, or importance, but rather are used to distinguish one element from another.

Certain terms are used herein and in the appended claims to refer to particular components. As one skilled in the art will appreciate, different persons may refer to a component by different names. This document does not intend to distinguish between components that differ in name but not function.

Also, the terms “including” and “comprising” are used herein and in the appended claims in an open-ended fashion, and thus should be interpreted to mean “including, but not limited to . . . ” Further, reference herein and in the appended claims to components and aspects in a singular tense does not necessarily limit the present disclosure or appended claims to only one such component or aspect, but should be interpreted generally to mean one or more, as may be suitable and desirable in each particular instance.

Preferred embodiments of the present disclosure thus offer advantages over the prior art and are well adapted to carry out one or more of the objects of this disclosure. However, the present disclosure does not require each of the components and acts described above and are in no way limited to the above-described embodiments or methods of operation. Any one or more of the above components, features and processes may be employed in any suitable configuration without inclusion of other such components, features and processes. Moreover, the present disclosure includes additional features, capabilities, functions, methods, uses and applications that have not been specifically addressed herein but are, or will become, apparent from the description herein, the appended drawings and claims.

The suffix “(s)” as used herein is intended to include both the singular and the plural of the term that it modifies, thereby including at least one of that term (e.g., the colorant(s) includes at least one colorants). “Optional” or “optionally” means that the subsequently described event or circumstance can or cannot occur, and that the description includes instances where the event occurs and instances where it does not. As used herein, “combination” is inclusive of blends, mixtures, alloys, reaction products, and the like.

The present invention now will be described more fully hereinafter with reference to the accompanying drawings, in which some preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

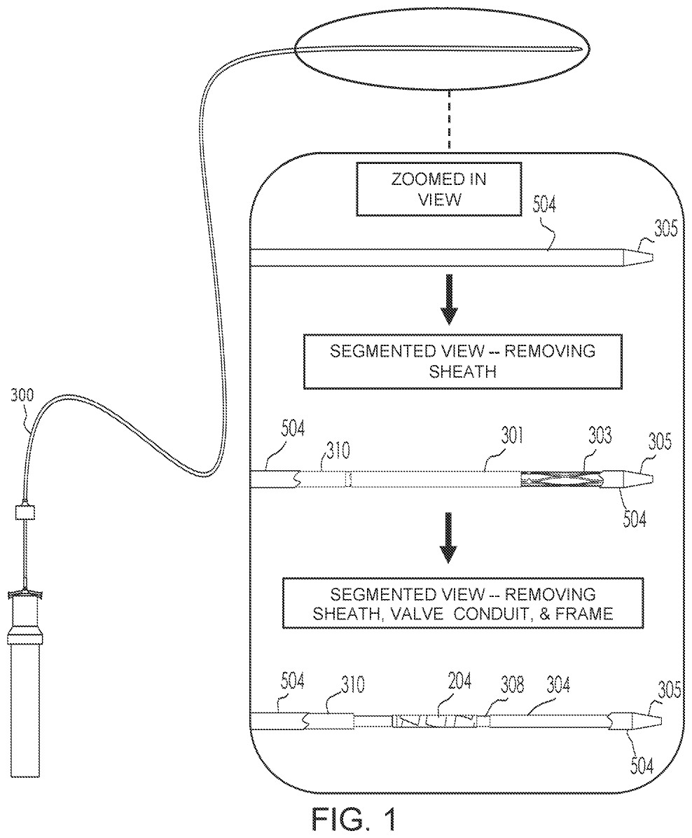

Referring to FIG. 1, the percutaneous endovascular centrifugal heart pump 300 is used to support the heart, including both the left side and the right side of the heart. As used herein, the term “percutaneous endovascular centrifugal heart pump 300” may be referred to herein simply as “heart pump 300” or “pump 300.” The application is directed through the motion of fluid from one location to another to support the human heart, providing blood supply to the body at a time when the native human heart is weak. Pump 300 may be introduced to the human body through the arterial system-arterial percutaneous endovascular centrifugal heart pump 300a, or through the venous system-venous percutaneous endovascular centrifugal heart pump 300b. As used herein, the term “arterial percutaneous endovascular centrifugal heart pump 300a” may be referred to herein simply as “arterial heart pump 300a” or “heart pump 300a” or “arterial pump 300a.” Similarly, as used herein the term “venous percutaneous endovascular centrifugal heart pump 300b” may be referred to herein simply as “venous heart pump 300b” or “heart pump 300b or venous pump 300b.”

Percutaneous heart pumps have a dichotomy—a smaller profile percutaneous heart pump may facilitate insertion and target a larger population; however, this leads to higher impeller speeds and flow velocity which may cause blood cell damage. On the other hand, larger percutaneous heart pumps have a difficulty in insertion and limits the population; however, these pumps may have lower impeller speeds, lower blood velocity, and minimize blood cell damage.

Therefore, the dichotomy is:

Table 1 below is a comparison between Impella CP [AbioMed, “Impella® 2.5, 5.0, LD and Impella CP® INSTRUCTIONS FOR USE & CLINICAL REFERENCE MANUAL for Use During Cardiogenic Shock Impella Ventricular Support Systems,” 2016 manufactured by AbioMed Company of Danvers, MA. www.abiomed.com.] and the present invention.

Thus, the present invention—the percutaneous endovascular centrifugal heart pump 300—solves this dichotomy by having a small insertion profile with the ability to operate at a larger profile. This targets a larger population by facilitating insertion access to the human body, and at the same time offers lower impeller speeds minimizing blood cell damage to the patient.

Preliminary results of the percutaneous endovascular centrifugal heart pump 300, show an output flow rate of 13 L/minute with zero head pressure and a flow rate of 9.5 L/minute with a head pressure of 80 mmHg.

### Impeller

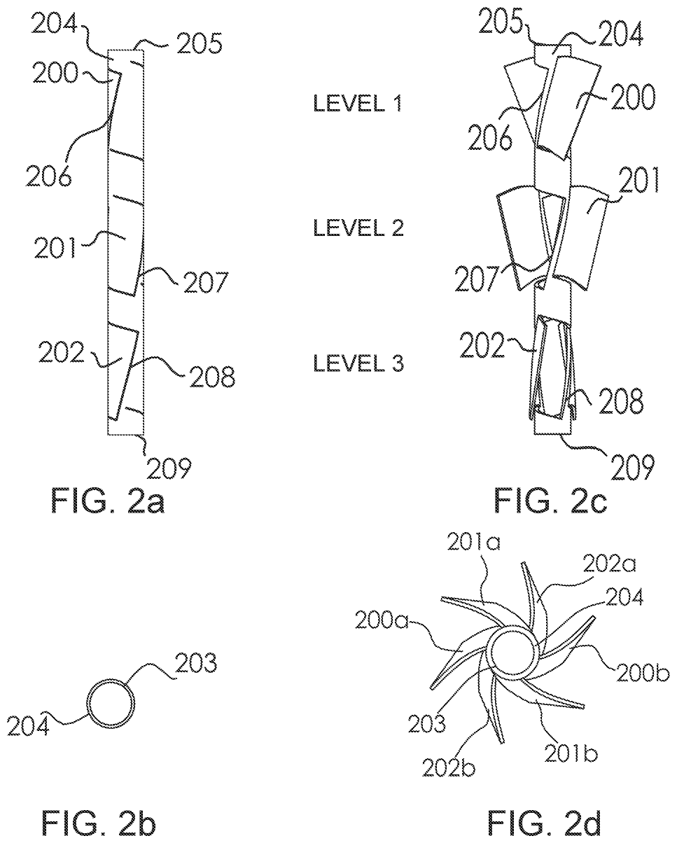

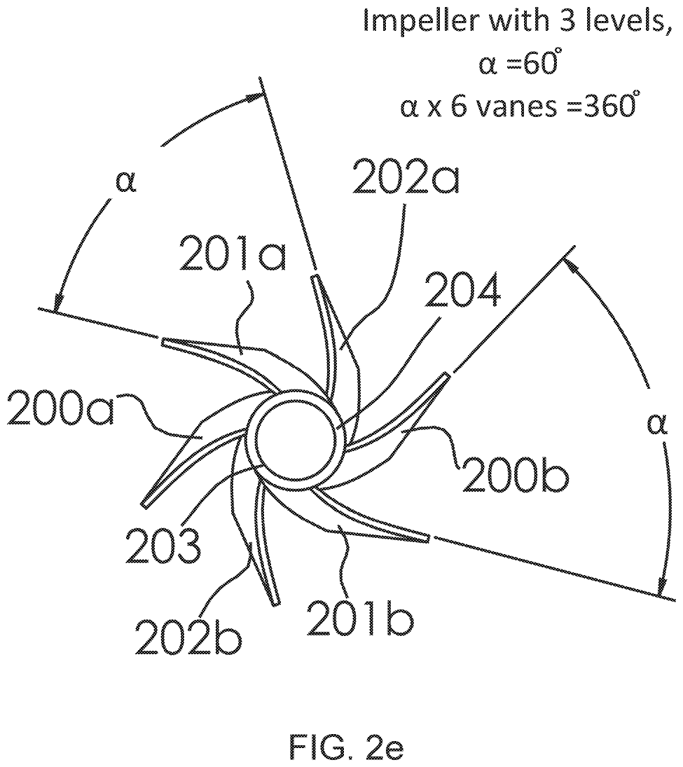

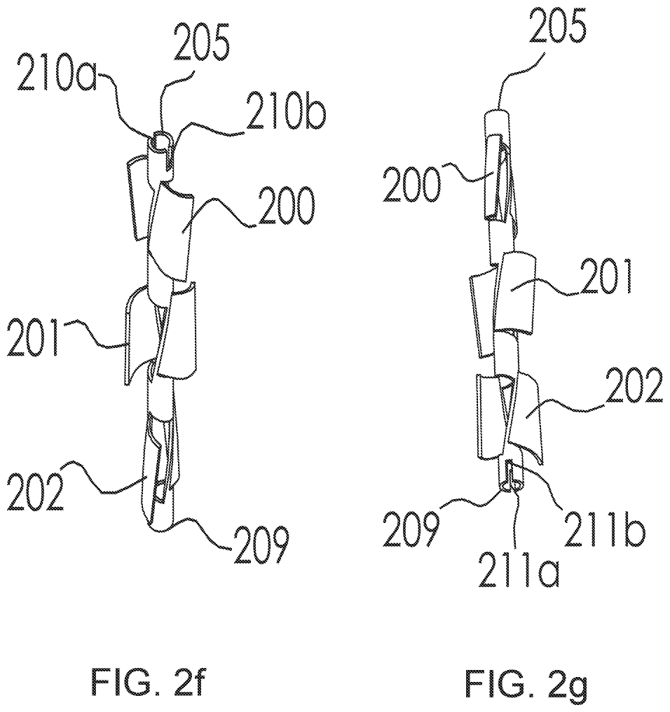

Referring now to FIGS. 2a-2g, one of the components that make up the percutaneous endovascular centrifugal heart pump 300 is the impeller 204. Impeller 204 is cut from a single sheet and or tube of smart material such as Nitinol (FIG. 2a and FIG. 2b). The diameter tube can range between about 1.5 mm to about 5 mm, preferably about 2.5 mm.

Impeller 204 includes an inner wall 203, impeller outflow end 205, and impeller inflow end 209. Impeller 204 includes a plurality of levels, such as a top-level vane 200, mid-level vanes 201, and lower-level vanes 202. Referring to FIG. 2c, the vanes are shape set to form. The Nitinol's austenite transformation finish temperature, Af, can range between about 10 C to about 40 C, preferably between about 5 C and about 20 C.

At each level there are two vanes spaced 180 degrees apart. Referring to FIG. 2d, top-level vane 200 includes two vanes 200a/200b, the mid-level 201 includes two vanes 201a/201b, and lower-level 202 includes two vanes 202a/202b. The tilted angulation of each vane from the horizontal plane may be in the range of 15 degrees to 65 degrees, preferably 35 degrees. This allows the diameter of impeller 204 to be between about 8 mm and about 20 mm, preferably about 14 mm.

Furthermore, each level of vanes shifted in proportion to the number of levels of impeller 204 includes. Thus, following the constitutive equation:

$$α = \frac{180}{L}$$

Where α is the insertion displacement or offset in degrees that each level will have from the previous vane insertion in degrees and L is the number of levels impeller 204 includes. For example, referring to FIGS. 2c and 2e, impeller 204 with three (3) levels results in 60 degrees angular displacement when viewed from the top view (180 divided by 3) (FIG. 2e). Thus, the angle between top-level 200a and mid-level 201a is 60 degrees, and the angle between mid-level 201a and lower-level 202a is also 60 degrees. The expanded state of impeller 204 can range between about 9 mm and about 22 mm, preferably about 15 mm.

The impeller 204 can return the vanes to the closed position when the device is resheathed as shown in FIG. 2a. That is, impeller 204 top-level vanes 200 will close to couple with top-level surface 206, mid-level vanes 201 will close to couple with mid-level surface 207, and lower-level vanes 202 will close to couple with lower-level surface 208.

Impeller 204 design is one of the features that permits percutaneous endovascular centrifugal heart pump 300 to function between about 4,000 RPMs and about 25,000 RPMs, preferably about 10,000 RPMs permitting it to pump more than about 5 L/min.

Referring to FIGS. 2f and 2g, Impeller 204 may include arterial slots 210a/210b for entry through the arterial system and venous slot 211a 211b for entry through the venous system.

The impeller 204 may also include a coating that can be hydrophobic or hydrophilic to minimize blood clot formation.

The impeller 204 may also include drug eluting capabilities to incorporate medication such as heparin to minimize blood clot formation.

### Frame

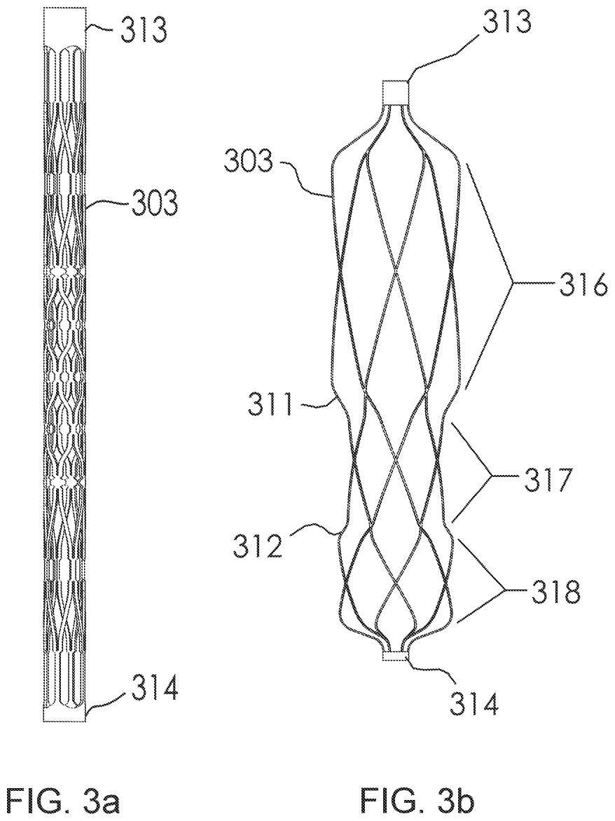

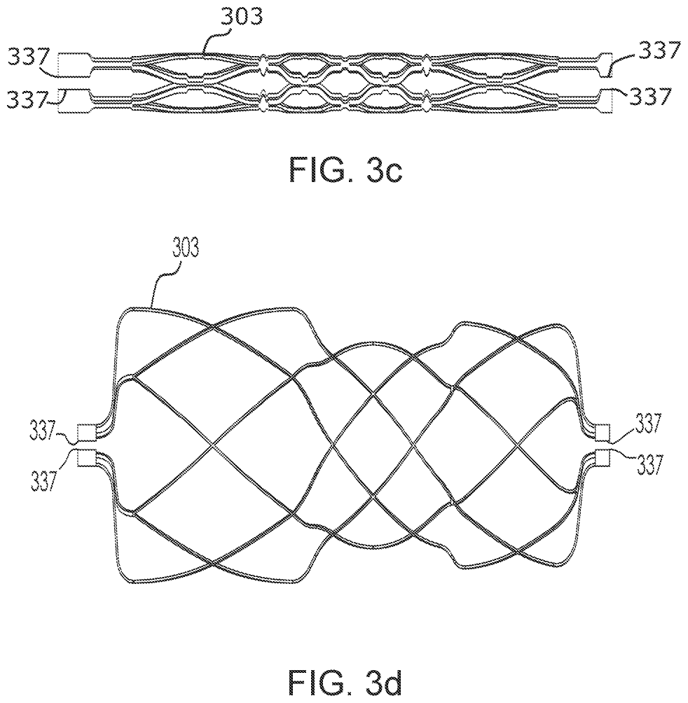

Referring now to FIGS. 3a-3d, percutaneous endovascular centrifugal heart pump 300 includes frame 303. Frame 303 is manufactured from a single tube of smart material, such as Nitinol, having a diameter between about 1 mm to about 5 mm, preferably between about 2 mm and about 3 mm. Frame 303 includes outflow section 313 and inflow section 314. Frame 303 may include one or two slits 337 to facilitate the insertion of a mandrel for shape setting frame 303 and for assembly (FIGS. 3c-d). Frame 303 can be shape-set (FIG. 3b), with an Af temperature that can range between about 10 C to about 35 C, preferably between about 15 C and about 20 C.

In its expanded state frame 303 may have a plurality of diameters. Referring to FIG. 3b, frame 303 may comprise three different diameters: top-section 316, mid-section 317, and lower-section 318. Top-section 316 may have an expanded diameter between about 10 mm and about 26 mm, with a preferred diameter of about 20 mm. Mid-section 317 may have an expanded diameter between about 9 mm and about 20 mm, with a preferred diameter of about 15 mm. Lower-section 318 may have an expanded diameter between about 10 mm and about 26 mm, with a preferred diameter of about 18 mm. These variations in diameters form valve anchors 311/312. Top valve anchor 311 is designed to attach to the upper section of the native leaflet and the lower valve anchor 312 is designed to attach to the lower section of the native heart valve leaflet. This design permits frame 303 to self-anchor. Anchors 311/312 aid in stabilizing the percutaneous endovascular centrifugal heart pump 300 during positioning and operation. These diameters are larger than prior art devices, and as a result, serve to more securely anchor the frame to the native valves leaflets prohibiting premature release or dislodgement.

In view of the design and the material used as discussed herein, the present invention provides for a small diameter percutaneous endovascular centrifugal heart pump during installation (between about 3 and about 4 mm-see Table 1 above) yet provides that the frame 303 can expand to preferably three different ranges of diameters depending on its top-section 316, mid-section 317, and lower-section 318 as noted above. This expansion is a significant improvement over the prior art by providing enhanced anchor points to the native valve leaflets as noted herein.

Thus, by selecting smart material, such as Nitinol, instead of a polymer as commonly used by the prior art, the present invention can be designed and shaped as discussed herein to be a small diameter yet once unsheathed, it can expand to the enhanced diameters for the three sections of the frame providing a firm anchor to the native leaflets and enhanced impeller size for improved flow within the within the valve conduit 301 as discussed herein.

Nitinol is known as a smart material or SMM or SMT. Nitinol is a nickel and titanium alloy and is used in the manufacture of vascular stents. The material is originally shaped into a predetermined form and then compressed and held in place by a sheath, for example. After it is placed in the desired location within the human body the sheath or other compressing means such as a wound wire is removed. The heat of the body then returns the material to its original shape.

Thus, in the present invention, frame 303 is initially shaped into a predetermined shape as shown for example in FIG. 3b or 3d to include anchoring points 311/312. It is then constricted or compressed and held in place as described herein and shown in FIGS. 3a and 3c. as described herein. Once heart pump 300a or 300b is positioned at the desired location within the heart, it is unsheathed and frame 303 is permitted to expanded to its predetermined shape having previously shaped anchor points 311/312. Frame 303 may include also include drug eluting capabilities to incorporate medication such as herapin to minimize blood clot formation.

### Arterial Percutaneous Endovascular Centrifugal Heart Pump Overview

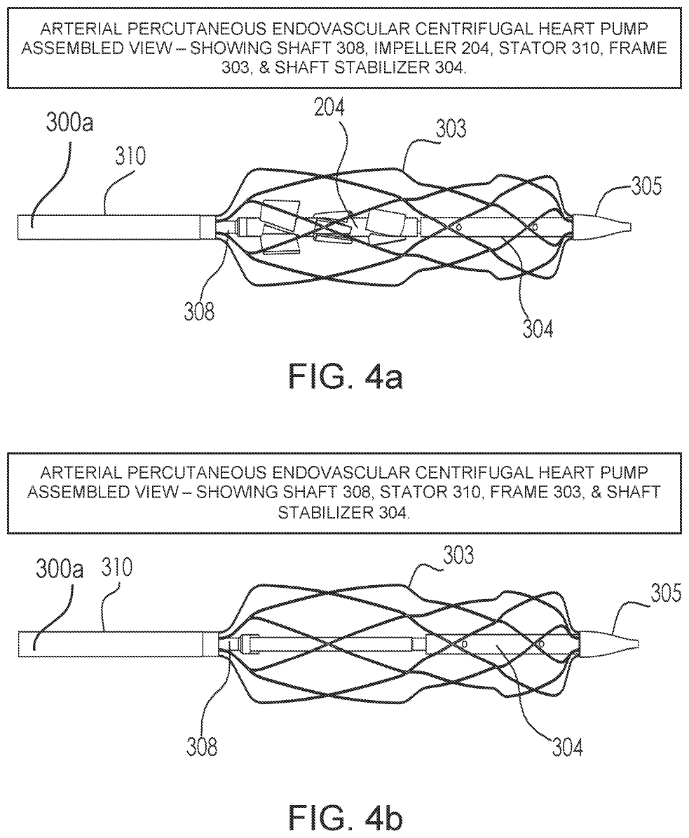

Referring now to FIGS. 4a and 4b, heart pump 300a includes stator 310, internal shaft 308, impeller 204, frame 303, shaft stabilizer 304, and insertion tip 305. During the operation of the present invention, impeller 204 and shaft 308 rotate, while the remaining components (stator 310, frame 303, shaft stabilizer 304, insertion tip 305) do not rotate. Stator 310 is attached to frame 303 and frame is attached to insertion tip 305 by bonding agent and or mechanical attachment. The bonding agent such be medical grade epoxy.

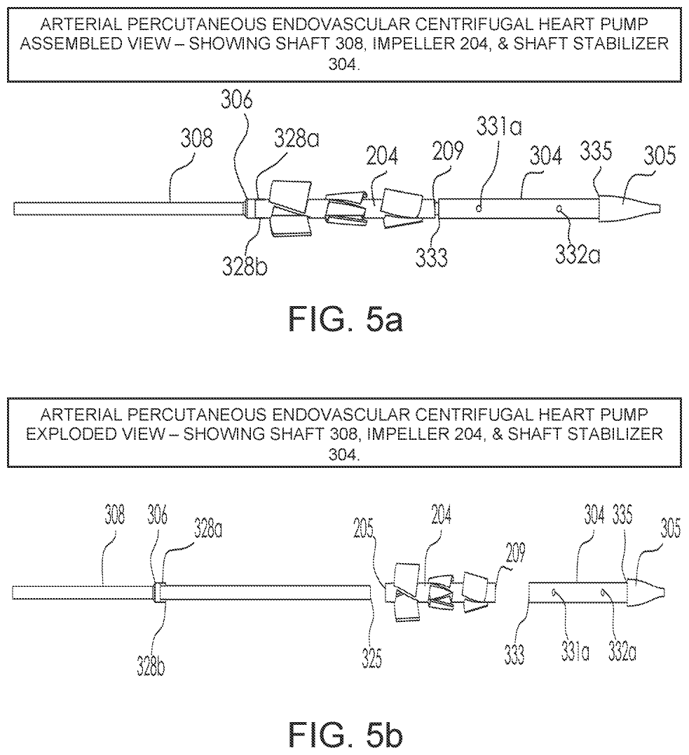

Referring now to FIGS. 5a and 5b, an impeller junction 306 is attached at each of its ends to impeller 204 and shaft 308. Impeller junction 306 includes two channels 328a/328b that insert along the impeller slots 210a/210b of the impeller 204. As such, impeller junction channels 328a/328b can provide guidance in the insertion of impeller 204 and aid in the transmission of rotational force to impeller 204. Furthermore, in the event the bonding of impeller 204 to shaft 308 fails, the impeller junction channels may act as a fixing mechanism to impeller 204.

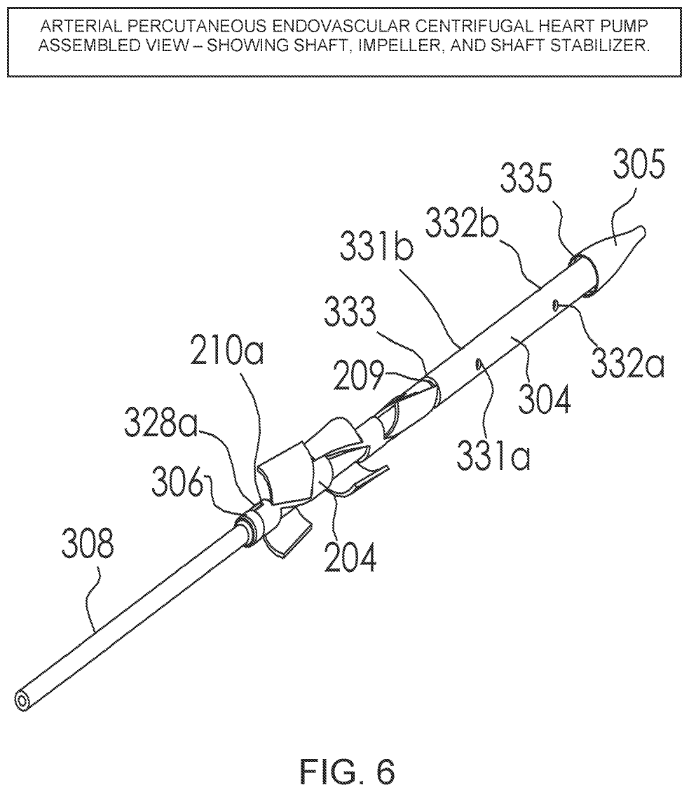

Referring now to FIGS. 5a, 5b and 6, the insertion of impeller 204 to shaft 308 is done by sliding the impeller outflow end 205 to the shaft distal end 325 all the way until it reaches the impeller junction 306 and the impeller junction channels 328a 328b are aligned and inserted with the impeller slots 210a 210b.

Referring now to FIGS. 3a-3d, 4a, 4b, 5a, 5b. and 6, frame 303 may have one or two slits 337 on either end, inflow section 314, and outflow section 313 allowing insertion of shaft 308 with impeller 204 for assembly. Frame 303 is attached to the stator 310 by bonding outflow section 313 to the distal end of stator 310. Furthermore, shaft stabilizer 304 is placed by inserting the distal end 325 in the shaft stabilizer entrance 333. The shaft stabilizer 304 is then attached to the frame 303 by bonding the frame's inflow section 314 to the shaft stabilizer frame attachment 335.

Referring to FIGS. 5a-5b and 6, shaft stabilizer 304 has heat and fluid dissipation ports in the proximal 331a/331b and in the distal 332a/332b. These ports may facilitate heat transfer and minimize stagnant flow between shaft 308 and shaft stabilizer 304. The shaft stabilizer heat dissipation proximal ports 331a/331b may be spaced 180 degrees apart. Furthermore, distal ports 332a/332b may also be spaced 180 degrees for the shaft stabilizer heat dissipation. The longitudinal spacing between ports 331a/331b and ports 332a/332b may range between about 5 mm and about 50 mm, preferably about 18 mm. The diameter of ports 331a/331b and 332a/332b may range between about 0.10 mm and about 3 mm, preferably about 1 mm.

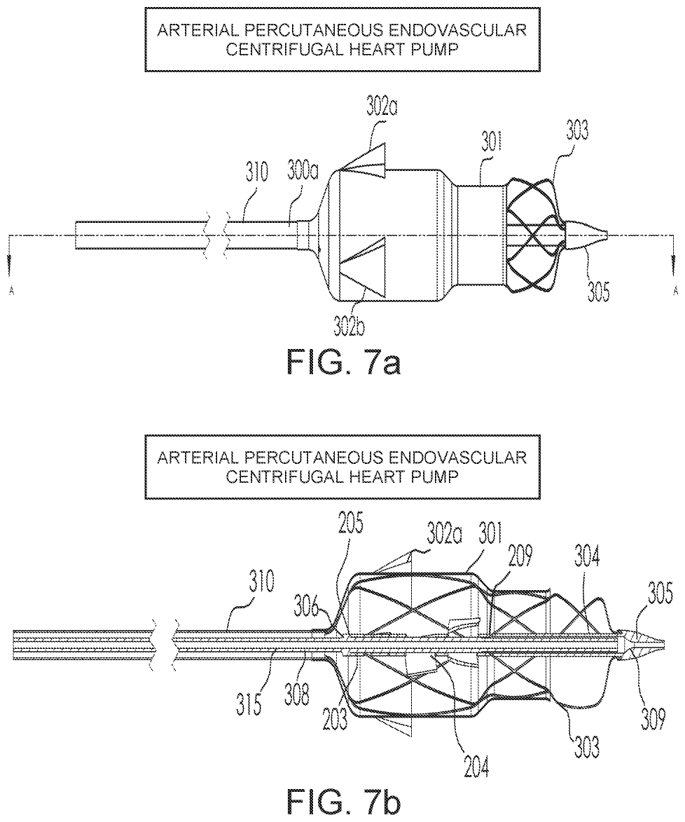

FIG. 7a illustrates a side view of arterial heart pump 300a having stator 310, valve conduit 301, valve conduit valves 302, frame 303, and insertion tip 305.

FIG. 7b illustrates a cross-sectional view of arterial heart pump 300a having impeller 204 and shaft 308.

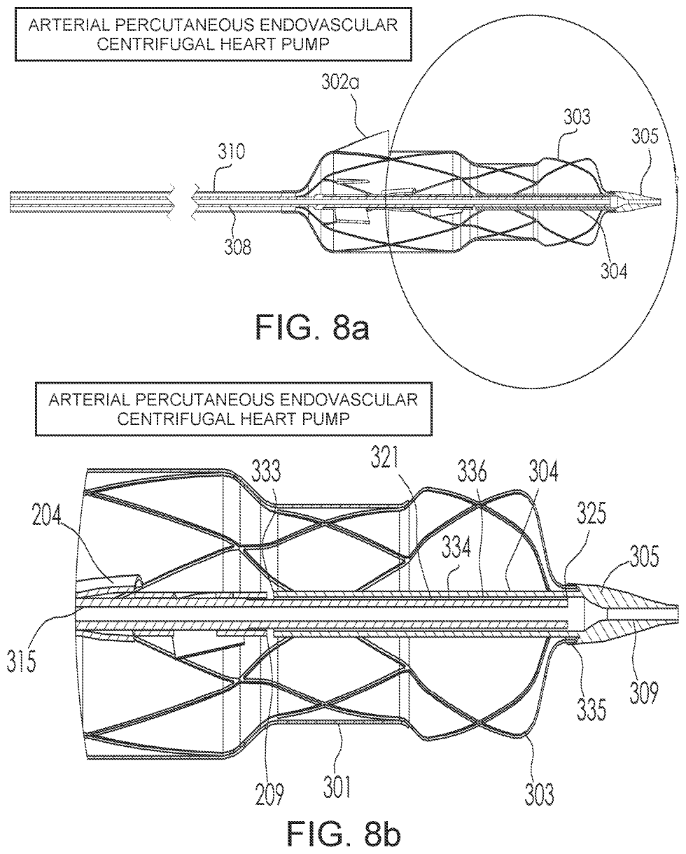

Referring now to FIGS. 8a and 8b, arterial heart pump 300a is illustrated showing the interactions of stator 310, shaft 308, impeller 204, frame 303, insertion tip 305, shaft stabilizer 304, valve conduit 301, valve conduit valves 302a. Frame 303 is attached to shaft stabilizer frame attachment 335. Shaft 308 is inside the shaft stabilizer inner wall 336. Shaft 308 is not fixed to the shaft stabilizer 304 and or the insertion tip 305. Shaft outer wall 321 and inner wall 336 of the shaft stabilizer forming an annular gap 334. Shaft stabilizer 304 has a clearance of between about 0.10 mm and about 1 mm between shaft stabilizer inner wall 336 and shaft outer wall 321. Shaft 308 includes an inner lumen 315, which allows a guidewire to pass through. The guidewire may exit inner lumen 315 at the insertion tip inner lumen 309.

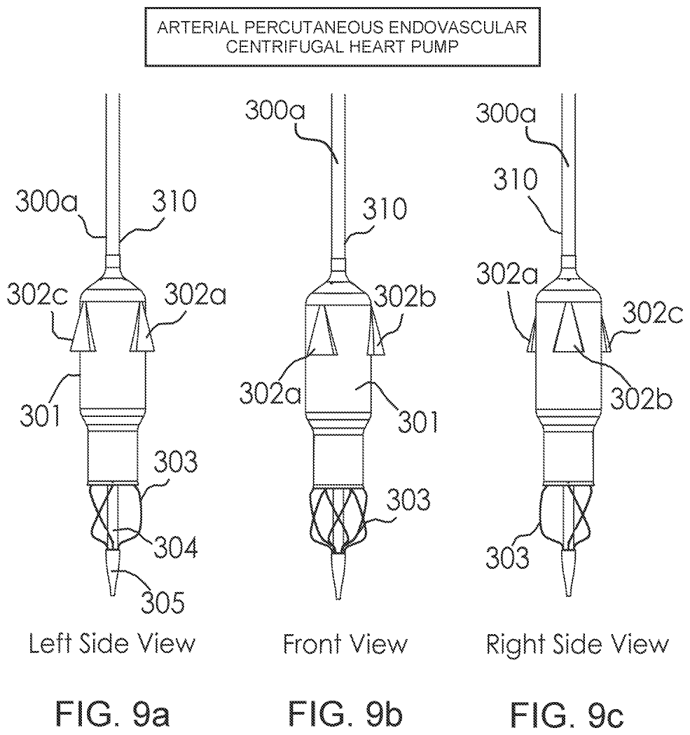

FIGS. 9a-9c illustrate the side view, front side view, right side view of the arterial percutaneous endovascular centrifugal heart pump 300a. As shown, 300a has stator 310 that is attached to frame 303 and contains valve conduit 301, valves 302a/302b/302c, frame 303, insertion tip 305, and shaft stabilizer 304. Valve conduit 301 may be made from biological material and synthetic material. Biological material such as ovine, bovine, or porcine pericardium is illustrated in FIGS. 9-11. Synthetic material may be Polytetrafluoroethylene (PTFE), expanded polytetrafluoroethylene (ePTFE), silicone, Polyethylene, polyurethane, and Nylon.

Referring still to FIGS. 9-11, valve conduit 301 may have a thickness that can range from about between 0.01 mm and 0.30 mm, preferably about 0.05 mm. Valve conduit 301 may be placed on the outside of frame 303, on the inside of frame 303, or on the outside and inside of frame 303. Valve conduit 301 may adapt to the geometry of frame 303, maintaining top section 316 and frame midsection 317 anchored at 311 and 312.

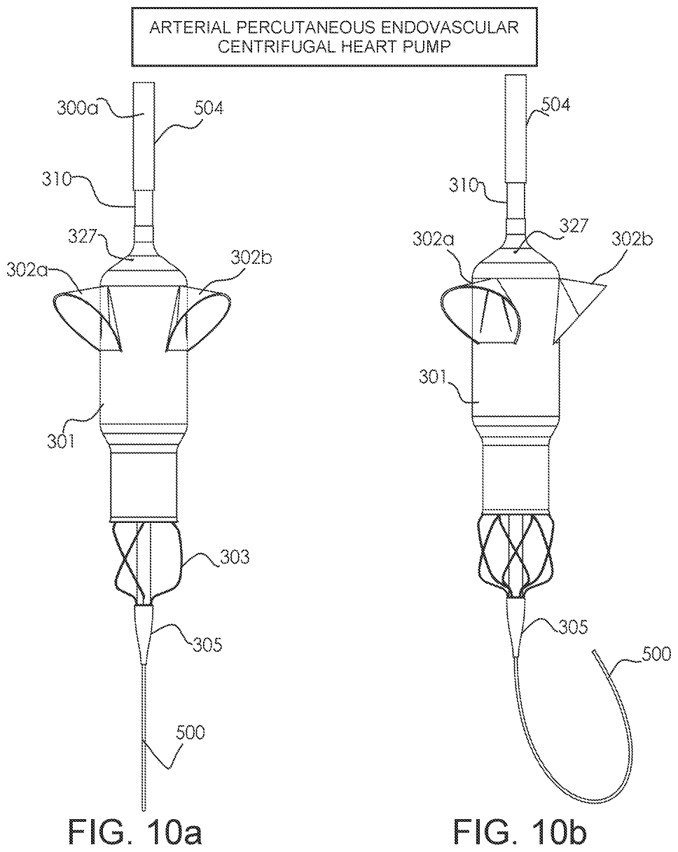

During insertion sheath 504 keeps valve conduit 301, valve conduit valves 302a/302b 302c, frame 303, and impeller 204 in a collapsed state. When sheath 504 is retracted, valve conduit 301, valve conduit valves 302a/302b/302c, frame 303, and impeller 204 expand as shown in FIGS. 10a and 10b. Sheath 504 requires enough tensile strength to contain said components collapsed. Sheath 504 may be manufactured of a polymer such as PTFE, FTP, ETFT, polypropylene or polyethylene, or combination of metal and polymer such metals as nitinol, stainless steel, cobalt chromium. Sheath 504 may have a diameter ranging from between about 1 mm to about 6 mm, preferably about 3 mm. Sheath 504 may have a wall thickness that ranges from between about 0.01 mm to about 0.6 mm, preferably about 0.25 mm.

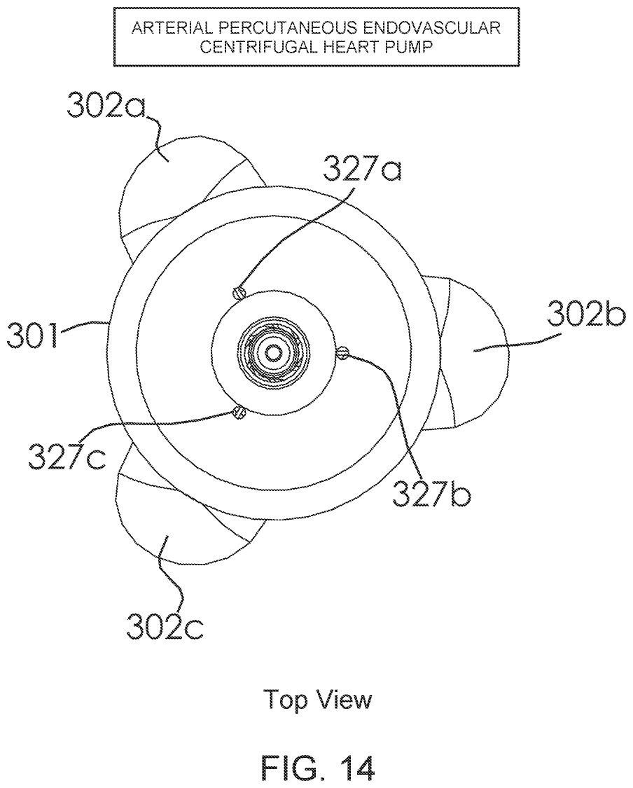

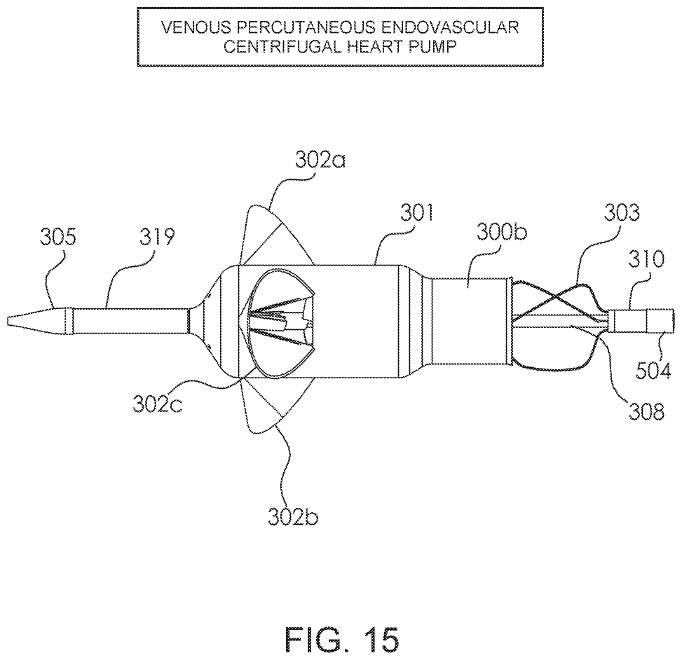

Referring still to FIGS. 9a-9c and 10a and 10b, conduit valves 302a/302b/302c may have a thickness that can range from between about 0.01 mm to about 0.35 mm, preferably about 0.05 mm. Valve 302 may comprise a plurality of valves such as 302a/302b/302c, though preferably two or three valves. Valves 302a/302b/302c may have limited openings as shown in FIG. 9a-9c or a fuller opening as shown in FIGS. 10a and 10b. Referring to FIG. 14, valve conduit 301 may also include circulation jets 327 that allow fluid to mobilize, minimizing stagnant flow.

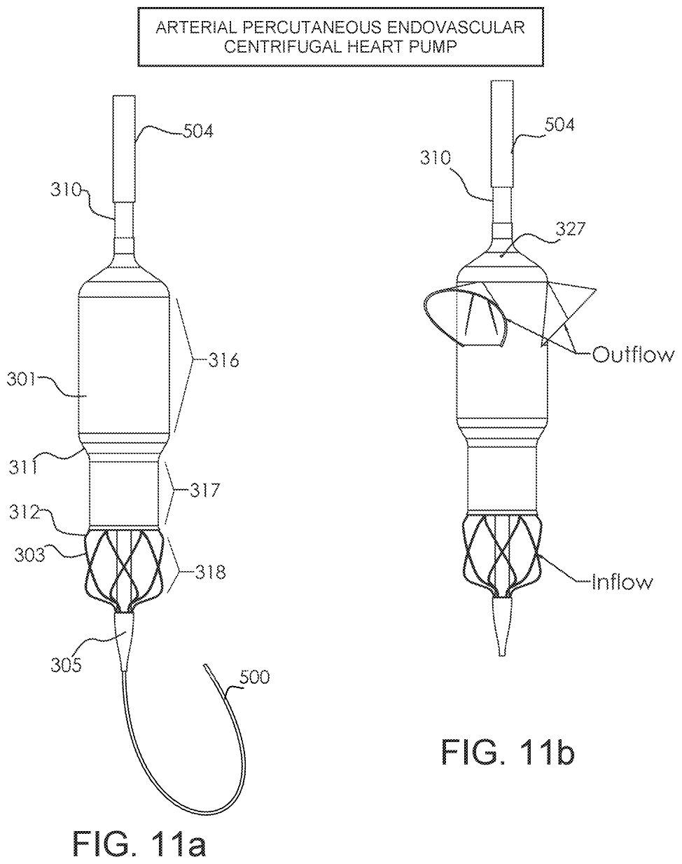

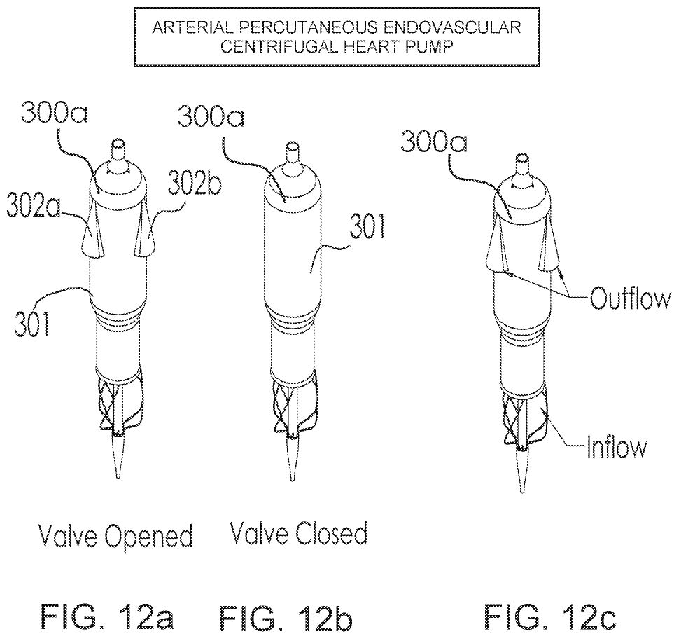

Valve conduit valves 302a/302b/302c are activated as a function of differential pressure, When the pressure within valve conduit 301 is greater than the pressure outside valve conduit 301, conduit valves 302a/302b/302c open as shown in FIGS. 10a-10b and 11b. However, when the pressure outside valve conduit 301 is greater than the pressure inside valve conduit 301, then valves 302a/302b/302c close preventing backflow as shown in FIG. 12b.

Arterial percutaneous endovascular centrifugal heart pump 300a suctions fluid from the inflow section of frame 303 and transitions the fluid across valve conduit 301 towards the outflow outside valves 302a/302b/302c as shown in FIG. 12c.

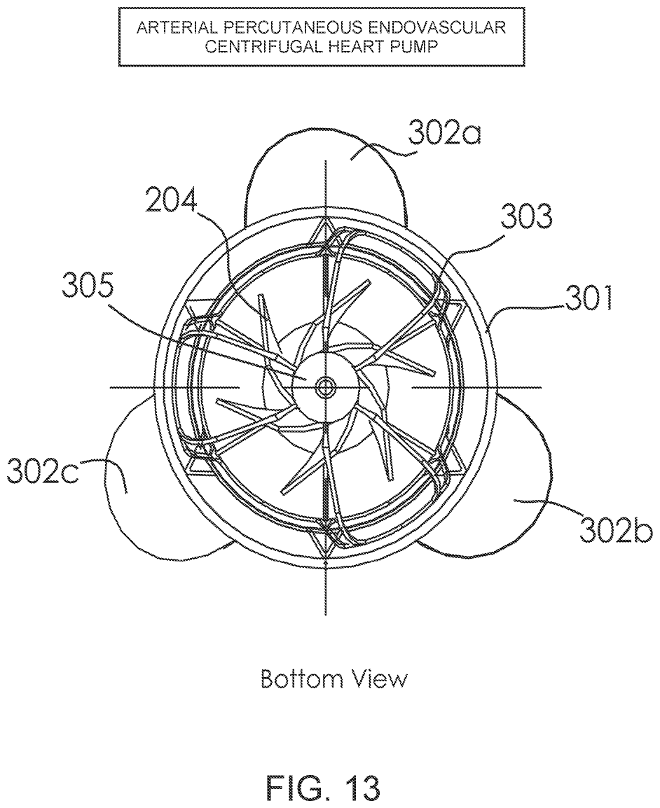

FIG. 13 is a bottom view of arterial percutaneous endovascular centrifugal heart pump 300a. It shows impeller 204 and valve conduit valves 302a/302b/302c. Furthermore, top view (FIG. 14) of arterial heart pump 300a illustrates valve conduit 301, valve conduit valves 302a/302b/302c, and conduit circulation jets 327a/327b/327c.

### Venous Percutaneous Endovascular Centrifugal Heart Pump Overview

Referring to FIG. 15, venous percutaneous endovascular centrifugal heart pump 300b may be inserted in the patient's body through the venous system. Venous percutaneous endovascular centrifugal heart pump 300b has sheath 504, frame 303, shaft 308, valve conduit 301, valve conduit valves 302a/302b/302c, venous shaft stabilizer 319, and insertion tip 305.

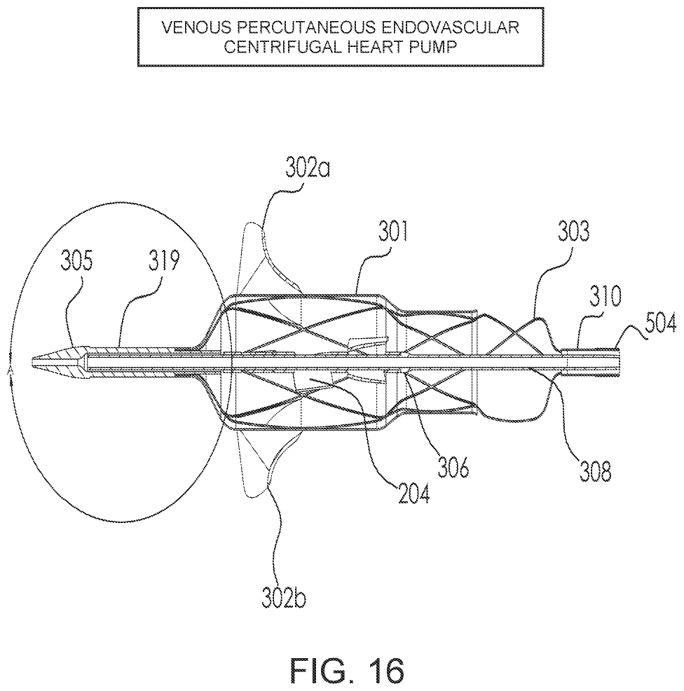

FIG. 16 shows a cross-section of the venous heart pump 300b, further illustrating sheath 504, stator 310, frame 303, valve conduit 301, impeller 204, conduit valves 302a/302b, venous shaft stabilizer 319, and insertion tip 305.

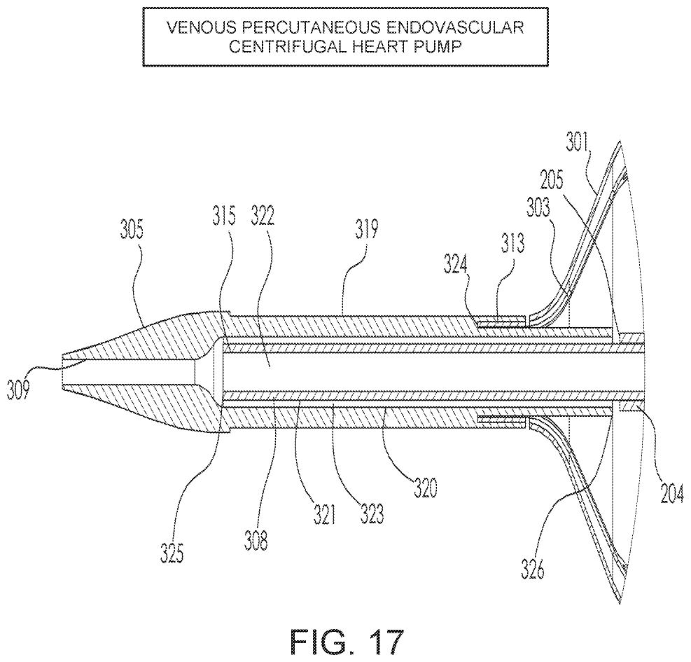

FIG. 17 is the detailed view A of FIG. 16 illustrating the interactions between the impeller 204, frame 303, venous shaft stabilizer 319, and insertion tip 305. Frame 303 is attached to venous shaft stabilizer frame slot 324. Outflow section 313 of frame 303 is inserted into the venous shaft stabilizer frame slot 324 and typically bonding using a medical grade epoxy or equivalent bonding agent. Shaft 308 (FIG. 16) is not attached to either frame 303 and or venous shaft stabilizer 319. When frame 303 is collapsed, shaft 308 does not move; however, frame 303 and venous shaft stabilizer 319 are displaced longitudinally relative to one another to allow frame 303 to reduce in diameter. When frame 303 is collapsed, venous shaft stabilizer inner wall 320 will advance across shaft outer wall 321 and outer wall 321 will thus remain within the inner wall 320 of the venous shaft stabilizer 319. When frame 303 expands, venous shaft stabilizer inner wall 320 moves longitudinally back across shaft outer wall 321. Thus, one purpose of venous shaft stabilizer 319 is to minimize and stabilize movement of shaft 308 as shaft 308 is rotating.

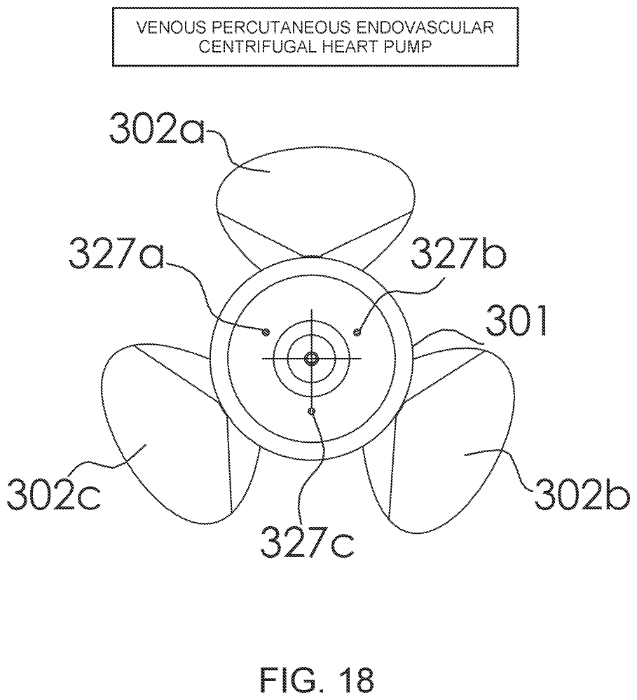

Referring now to FIG. 18, a top view of venous heart pump 300b is shown illustrating valve conduit 301, valves 302a/302b/302c, and valve conduit recirculating flow jets 327a/327b/327c. Recirculating flow jets 327a/327b/327c serve to minimize stagnant flow. Recirculating flow jets 327a/327b/327c are preferably apertures that allow fluid to move as function of pressure. The diameter of each aperture 327a/327b/327c may vary between about 0.1 mm and about 3 mm, preferably about 0.5 mm. Each valve conduit 301 includes one or more recirculating flow jets 327a/327b/327c.

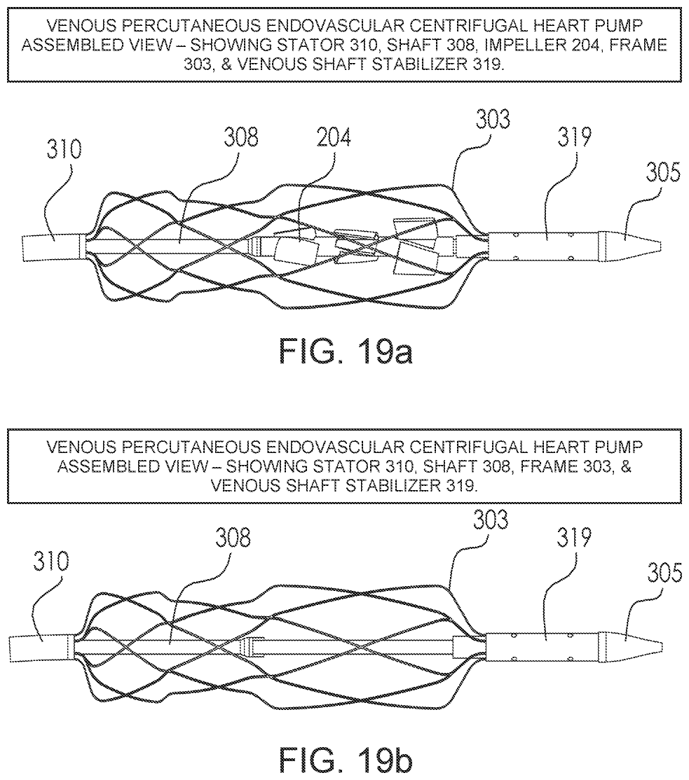

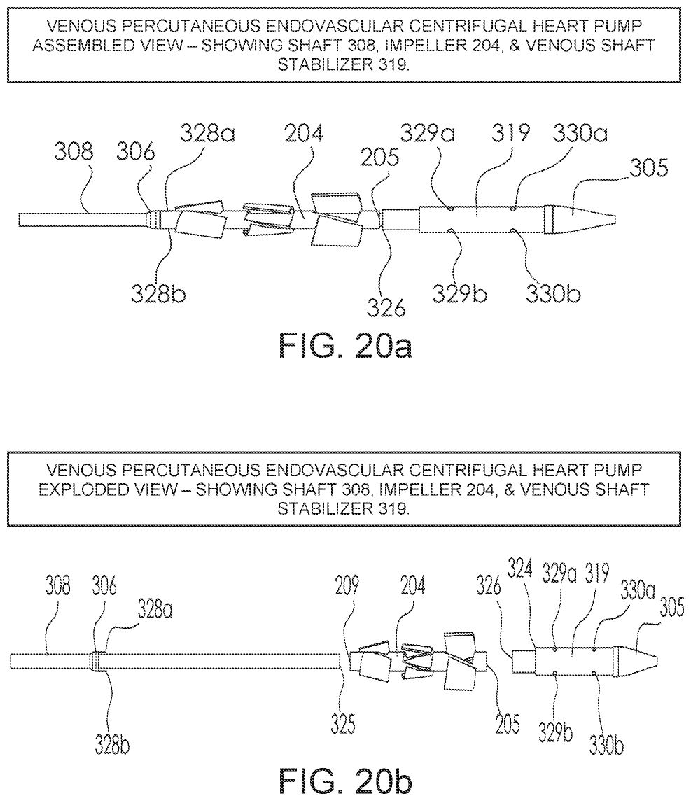

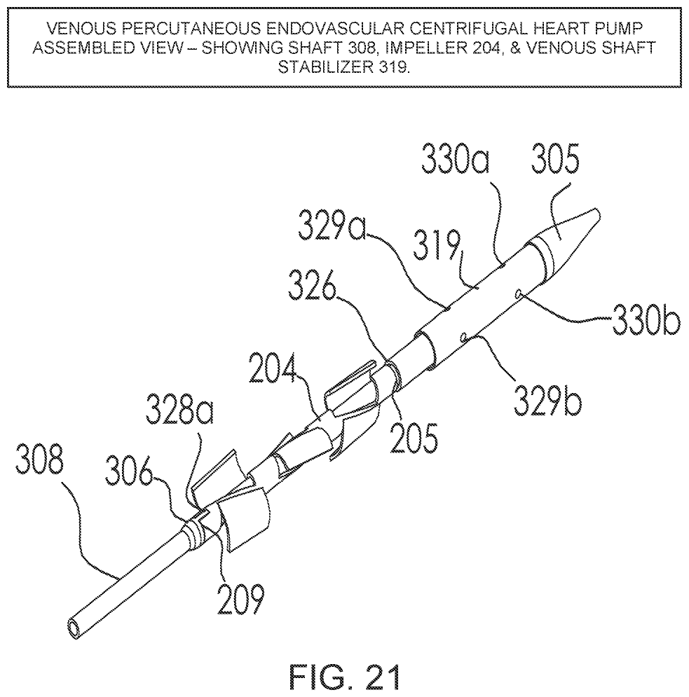

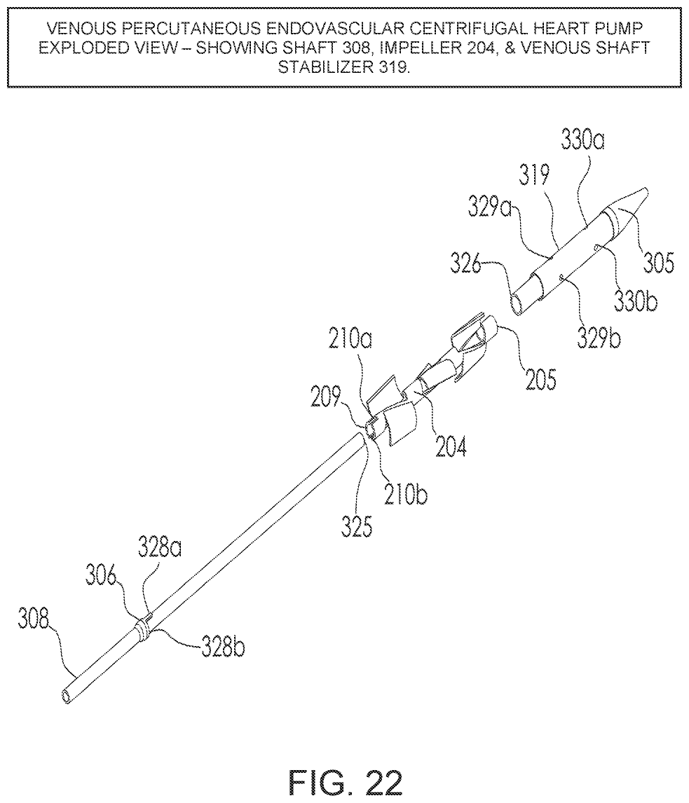

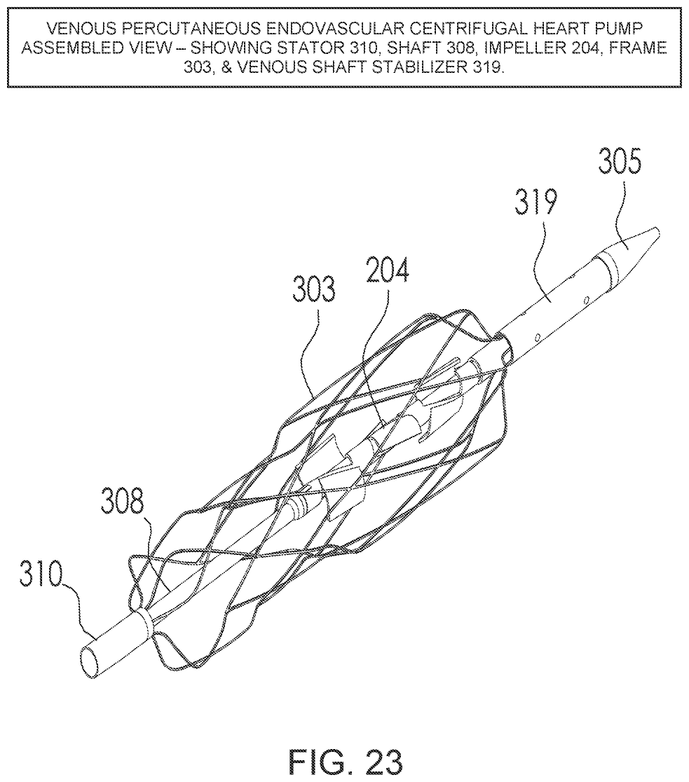

Referring now to FIGS. 19a-19b, 20a-20b, 21, 22, and 23, impeller 204 is attached at its proximal end to shaft 308 and at its distal end to shaft distal end 325. Impeller slides 210a/210b are aligned with shaft impeller channel 328a/328b. Impeller inflow end 209 aligns with impeller junction 306. Impeller 204 is bonded to the shaft 308, using a medical grade epoxy or equivalent bonding agent. The shaft 308 rests inside the venous shaft stabilizer 319, and the impeller outflow end 205 faces venous shaft stabilizer entrance 326. During the operation of the venous pump 300b, impeller 204 and shaft 308 rotate, while the remaining components (stator 310, frame 303, venous shaft stabilizer 319, insertion tip 305) do not rotate. The venous shaft stabilizer 319 has heat and fluid dissipation ports in the proximal 329a/329b and in the distal 330a/330b. These ports may facilitate heat transfer and minimize stagnant flow between shaft 308 and venous shaft stabilizer 319. The venous shaft stabilizer heat dissipation proximal ports 329a/329b may be spaced 180 degrees apart. Furthermore, distal ports 330a/330b may also be spaced 180 degrees for the shaft stabilizer heat dissipation. The longitudinal spacing between ports 329a/329b and ports 330a/330b may range between about 5 mm and about 50 mm, preferably about 18 mm. The diameter of ports 329a/329b and 330a/330b may range between about 0.10 mm and about 3 mm, preferably about 1 mm.

### Overview of Arterial Pump 300a Versus Venous Pump 300b

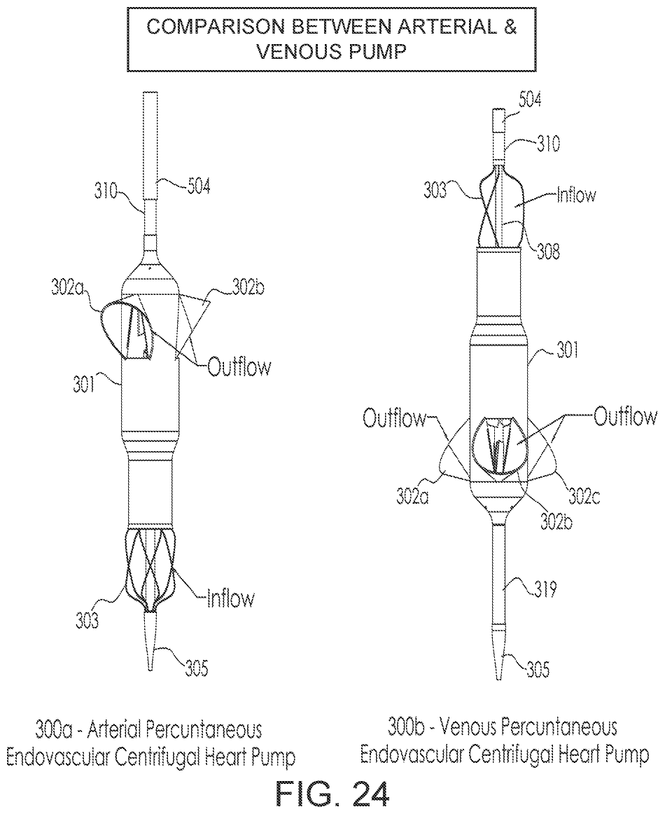

Referring to FIG. 24, a comparison between the arterial percutaneous endovascular centrifugal heart pump 300a and the venous percutaneous endovascular centrifugal heart pump 300b is shown. The inflow and outflows are reversed for the venous percutaneous endovascular centrifugal heart pump 300b compared to the arterial percutaneous endovascular centrifugal heart pump 300a as well as valve conduit 301, frame 303, valve conduit valves 302a/302b/302c. Furthermore, venous shaft stabilizer 319 is extended on the venous heart pump 300b compared to the shaft stabilizer 304 of the arterial device 300a.

### Motor Connection

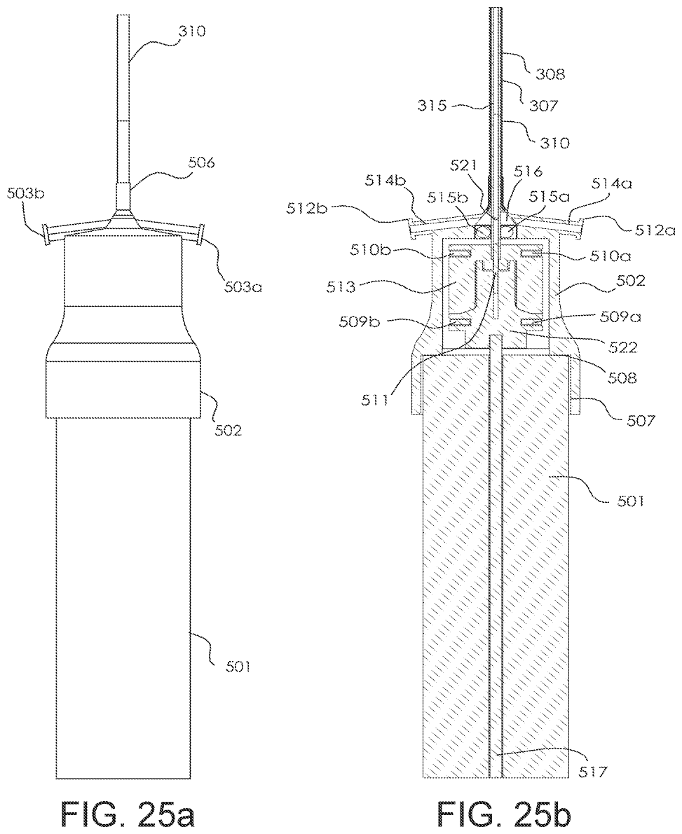

Referring now to FIGS. 25a and 25b, motor 501 drives heart pump 300. Stator motor connector 502 attaches stator 310 and shaft 308 and shaft 308 to motor 501. Connector 502 may include flushing ports 503a/503b. Ports 503a/503b are used to add or remove fluid from inside stator 310 and shaft 308. Luer lock 512a/512b may be used to achieve this connection. Ports 503a/503b may be connected to a continuous fluid infusion pump to lubricate the system mitigating frictional and vibrational forces.

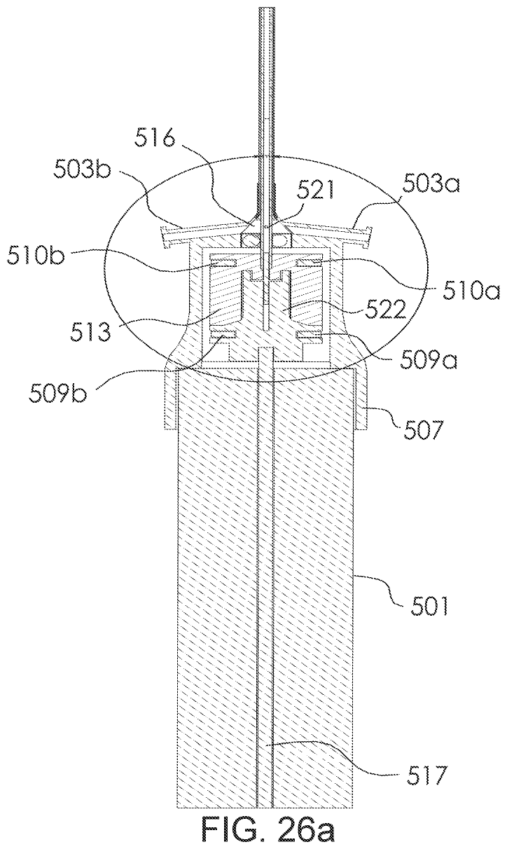

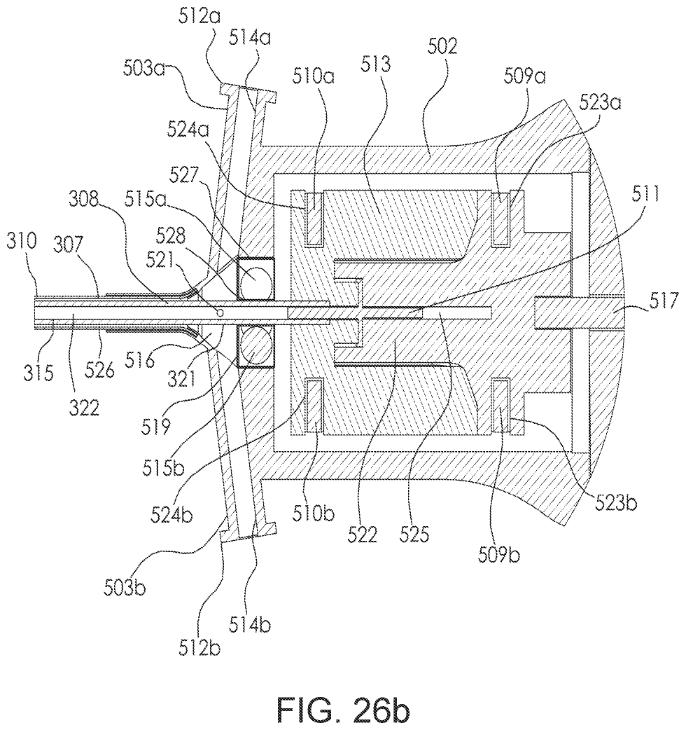

Referring still to FIGS. 25a-b but now also to FIGS. 26a-b, 27a-b, and 28, motor 501 may include motor shaft 517 which will drive shaft 308. Preferably, motor shaft 517 is attached to motor junction 522 by either fasteners or a by bonding, thereby fixing motor junction 522 to motor shaft 517. Motor junction 522 may include one or more magnets 509a/509b/509c/509d. Motor junction 522 may have magnet slots 523a/523b/523c/523d wherein magnets 509a/509b/509c/509d may be inserted. Rotating junction 513 is attached to shaft 308 by means of chemical bonding or electromagnetic forces. Rotating junction 513 may include magnets 510a/510b/510c/510d within magnet slots 524a/524b/524c/524d. Magnet slots 523d and 524d are not shown in the figures due to their location on the opposite side of the figure.

Referring still to FIGS. 25a-b, 26a-b, 27a-b, and 28, shaft 308 is attached to bearing 519 (FIG. 26b) by attaching shaft outer wall 321 to the inner race 528 of bearing 519. Again, the attachment may be accomplished by bonding using medical-grade epoxy or an equivalent agent. Bearing 519 allows shaft 308 to rotate while stator motor connector 502 remains stationary. Bearing 519 includes ball bearings 515a/515b that allows the shaft 308 to rotate while maintaining stator motor connector 502 stationary. A seal 529 that prevents fluid from escaping or leaking past the bearing. Bearing 519 includes an outer race 527 that does not rotate and is attached to stator motor connector 502. The bearing 519 has ball bearings 515a/515b/515c/515d that may rotate to allow inner race 528 to rotate while outer race 527 remains stationary. This allows shaft 308 to rotate while stator motor connector 502 and stator 310 remain stationary.

Referring to FIGS. 25a-b and 26a-b, stator motor connector 502 is attached to stator 310. Shaft 308 includes a guidewire port 521 to allow fluid to enter shaft inner lumen 322, minimize stagnant flow, as well as provide lubrication to the guidewire. Fluid can be inserted or removed through flushing ports 503a/503b. Fluid enters or exits through flushing port lumen 514a/514b and enters the lubricating region 516. Fluid can enter guidewire lubrication port 521 and shaft-stator gap 526. Gap 526 is the space between shaft outer wall 321 and stator inner lumen 307. Rotating junction 513 has an inner lumen 525 that connects to shaft inner lumen 322 and allows the guidewire to enter or exit. This is done by the guidewire 500 entering the rotating junction inner lumen 525 followed by shaft inner lumen 322. Rotating junction inner lumen 525 is sealed by guidewire seal device 511 when motor junction 522 is inserted. Guidewire seal 511 may be made of a polymer such as silicone, nylon, PTFE; or metal such as stainless steel, cobalt-chromium; or a combination of a polymer and metal. Guidewire seal 511 is attached to motor junction 522. Thus, when motor junction 522 is connected to rotating junction 513, the rotating junction inner lumen 525 is sealed.

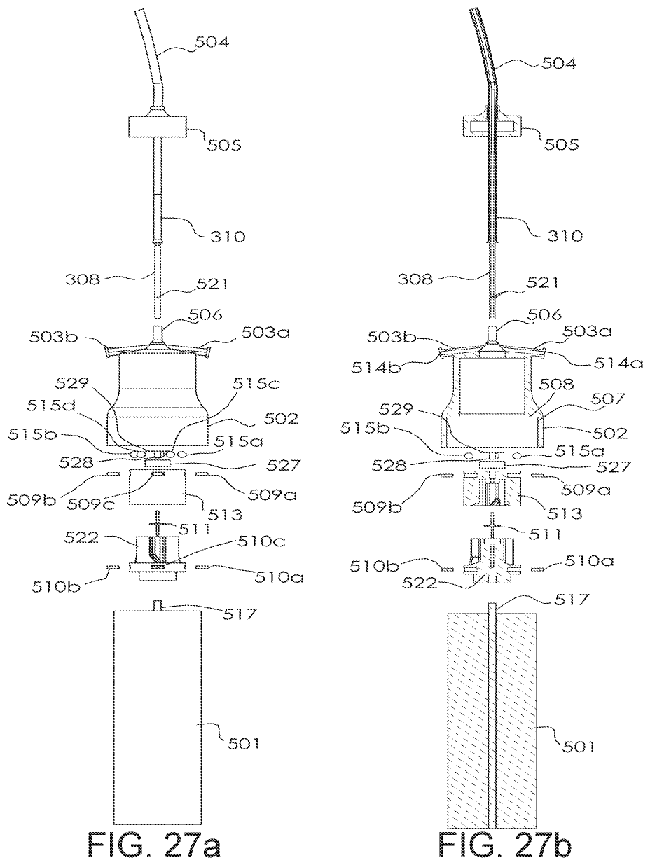

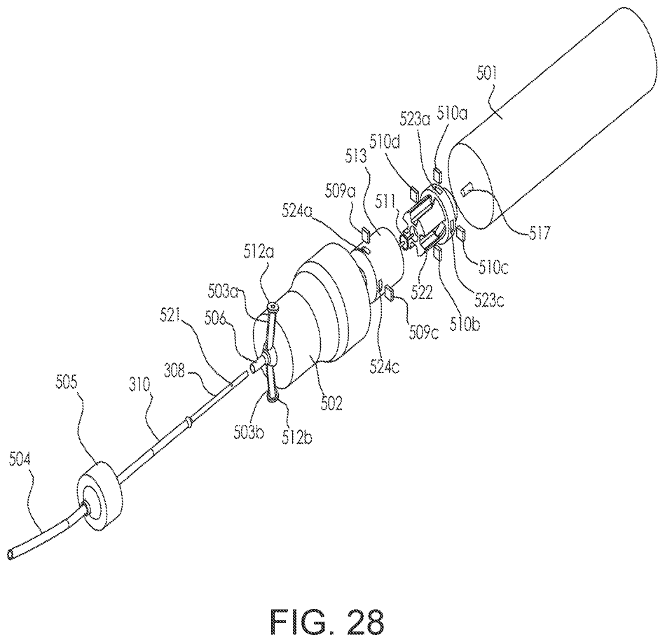

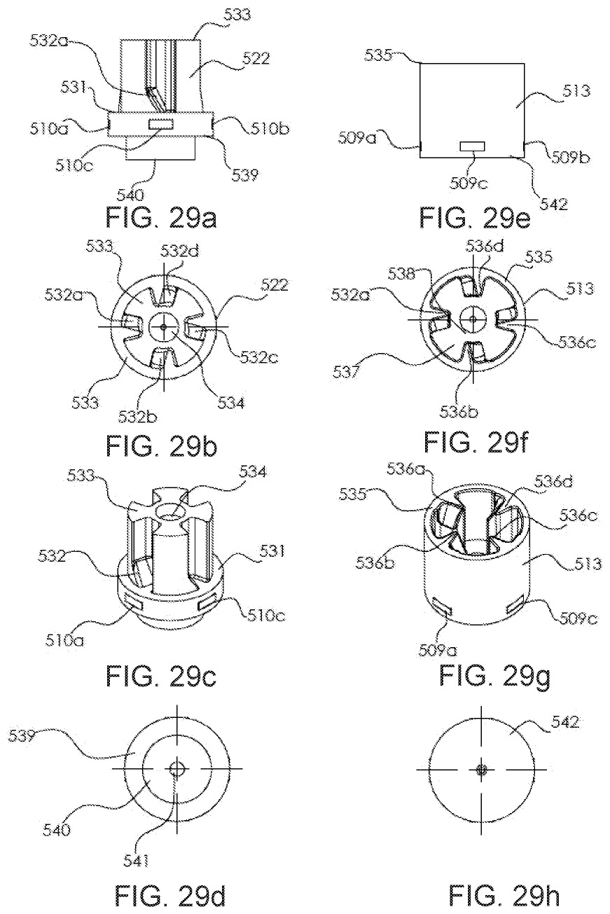

Referring to FIGS. 27a-b, 28, and 29a-h, motor junction 522 is attached to motor5 501. Rotating junction 513 is attached to the stator motor connector 502. Rotating junction 513 includes connecting guides 536a/536b/536c/536d that minimize the area, thus facilitating insertion to motor junction top surface 533 (FIGS. 29a-h). This is further guided by motor junction slope 532a/532b/532c/532d. The connection is further facilitated by motor junction magnets 509a/509b/509c/509d and rotating junction magnets 510a/510b/510c/510d. The magnets are placed so that motor junction 522 and rotating junction 513 are attracted to one another by placing opposite poles of the magnets at each junction. For instance, motor junction magnet 509a may have a north pole, while the rotating junction magnet 510 may have a south pole, thus creating an attraction force. This setup may be repeated for the remaining motor junction magnets 509b/509c/509d and the remaining rotating junction magnets 510b/510c/510d. This magnetic attraction locks the motor junction to the rotating junction, thus mating motor junctions' top surface 533 and the rotating junction's inner section 537.

### Unsheathing, Repositioning, Recapture, and Control Release Process

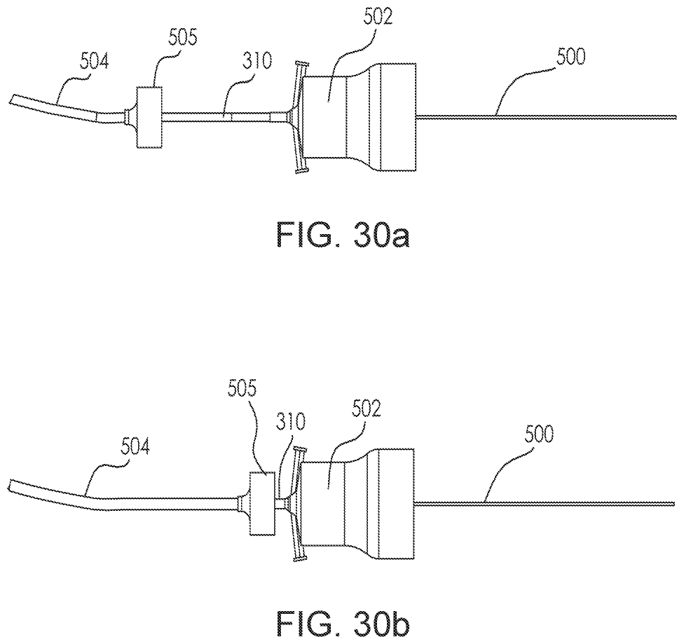

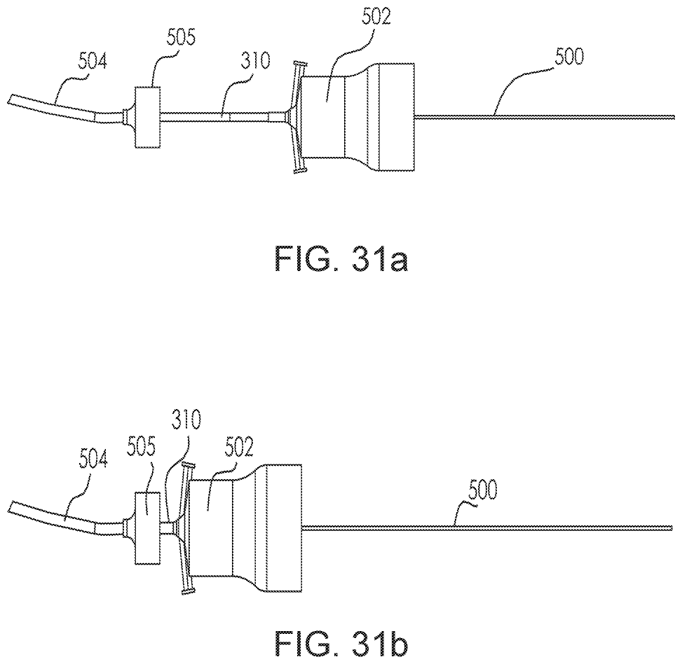

Referring now to FIGS. 30a, 30b, 31a, 31b, for the unsheathing, sheath valve adapter 505 and sheath 504 may be pulled toward stator motor connector 502 along stator 310, thus bringing sheath valve adapter 505 towards stator motor connector 502 as illustrated in FIG. 30b. Such a pushing force may be accomplished by pushing stator motor connector 502 towards sheath valve adapter 505 and sheath 504 along stator 310. This brings stator motor connector 502 towards sheath valve adapter 505 (FIG. 31a-b). Both these actions unsheath valve conduit 301 and impeller 204 in a controlled release manner using radiological angiographic and ultrasonic guidance. In the event the deployment is not satisfactory, the device may be recaptured and repositioned.

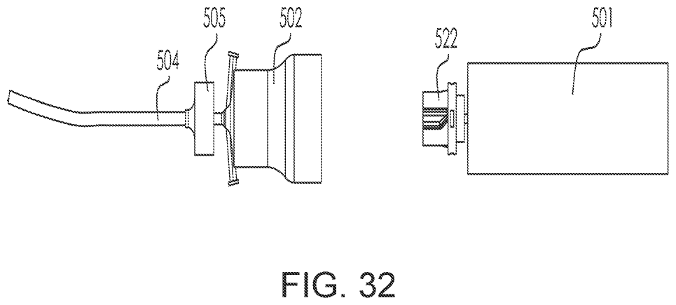

Referring to FIG. 32, when unsheathing is complete, guidewire 500 may be removed. At this point motor 501 is connected to stator motor connector 502 at motor junction 522. Motor 501 is joined with stator motor connector 502 using the rotating junction magnets 510a-d and motor junction magnets 509a-d.

To resheath or recapture the percutaneous endovascular centrifugal heart pump 300, sheath valve adapter 505 and sheath 504 are pulled away from stator motor connector 502 along stator 310. This can be done by either pushing or pulling relative to the different components described.

### Insertion of Arterial Percutaneous Endovascular Centrifugal Heart Pump

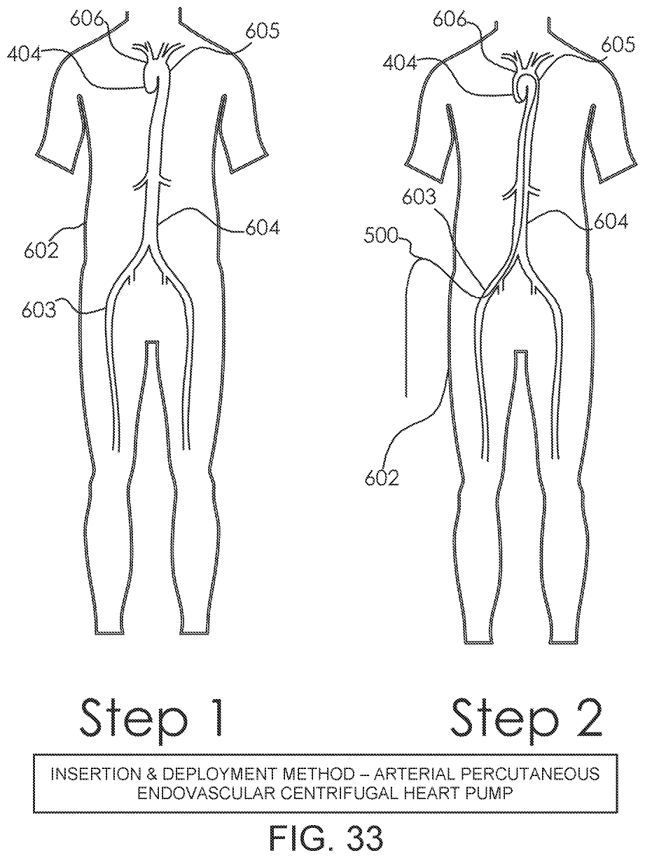

The insertion of the arterial percutaneous endovascular centrifugal heart pump 300a into the human body 602 may be summed up in five steps as shown in FIGS. 33-35.

Step 1 as shown in FIG. 33 is assessing the human body 602 by identifying access to the patient. Access is initially done through the femoral artery 603. Alternative access will be brachial or axillary artery or direct aortic puncture in cases that may need the present invention while performing open heart surgery.

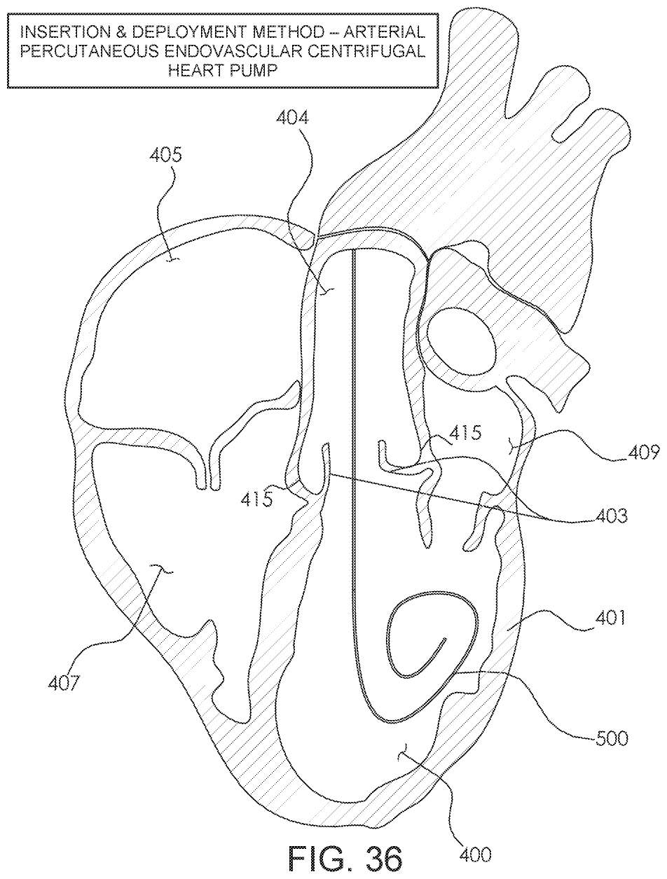

Step 2 is to insert a guidewire 500 into the femoral artery 603 and advance across the abdominal aorta 604 towards the descending aorta 605, crossing the aortic arch 606 and passing through the ascending aorta 404 into the left ventricle 400 (See also FIG. 36).

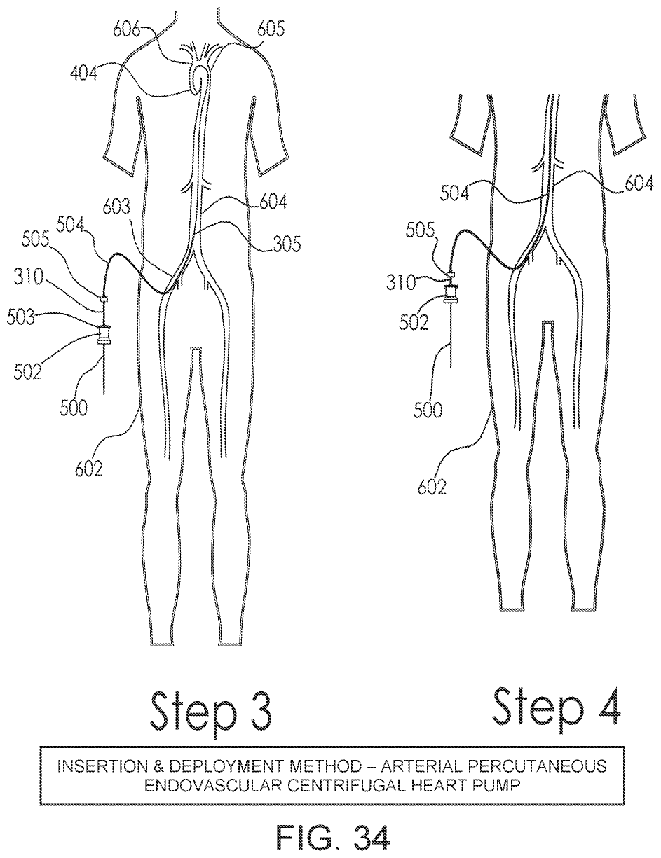

Step 3 (FIG. 34)—once the guidewire 500 is in place, the arterial percutaneous endovascular centrifugal heart pump 300a is advanced by inserting the guidewire 500 in the insertion tip inner lumen 309. The insertion tip 305 is advanced in the human body 602 by penetrating the skin into the femoral artery 603. The arterial percutaneous endovascular centrifugal heart pump 300a is further advanced to the abdominal aorta 604, desscending aorta 605, aortic arch 606 ascending aorta 404, crossing the aortic valve leaflets 403.

Step 4 (FIG. 34)—once the arterial percutaneous endovascular centrifugal heart pump 300a is unsheathed, by either pulling or pushing the sheath 504 and stator motor connector 502 together, and placing it across the aortic valve leaflets 403, the guidewire 500 may be removed. Once the guidewire 500 is removed, the motor 501 may be connected to the arterial percutaneous endovascular centrifugal heart pump 300a by means of the stator motor connector 502.

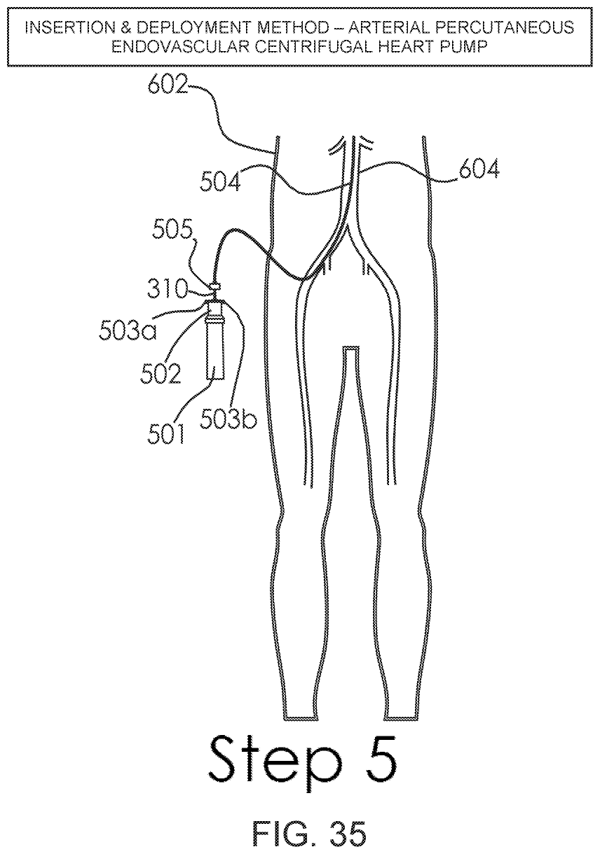

Step 5 (FIG. 35)—once the motor 501 is connected to the stator motor connector 502, the motor may be turned on which will turn the motor shaft 517, as well the shaft 308 and impeller 204. This will drive the blood from the left ventricle 400 across the valve conduit 301 towards the ascending aorta 404 unloading the left ventricle 400.

Vascular access is obtained using anatomical landmarks, radiological landmarks and ultrasound guided vascular access. The objective is to access the artery in the anterior wall of the vessel. For femoral access the goal is to access the femoral artery 603 above the bifurcation and below a tangential line traced at the superior border of the femoral head. For axillary artery, the plan is to access the vessel in the superior third of the humeral bone using anatomical landmarks, radiological landmarks and ultrasound guided access, taking special care not to interfere with the brachial plexus.

The artery will be accessed using the Seldinger technique, where a needle is advanced from the skin toward the vessel. When the needle is inside the vessel, a wire is advanced into the artery. The needle is withdrawn and a insertion sheath is advanced over the wire into the artery. Once vascular access is achieved, the insertion sheath is suctioned and flushed. A pigtail catheter is advanced over the wire into the left ventricle 400. The wire is removed, and the catheter is suctioned and flushed. Anticoagulation is started to achieved ACT levels between 250 to 300. Left ventricular pressure is recorded.

Guidewire 500 (preferably about 0.035 inches in diameter) is then advanced inside the pigtail catheter into the left ventricle 400 preferential. Once the guidewire 500 is placed in the left ventricle 400 the pigtail first and then the insertion sheath is removed from the body 602 the arterial percutaneous endovascular centrifugal heart pump 300a is advance into the left ventricle 400 over the guidewire 500. When the collapsed valve conduit 301 segment of the sheath 504 is across the aortic valve leaflets 403, the sheath 504 is retracted over the stator 310 or the stator 310 is advanced over the guidewire 500 unsheathing the valve conduit 301 with the impeller 204 inside. Once the valve conduit 301 is fully expanded across the aortic valve leaflets 403, the guidewire 500 is withdrawn from the body 602 and the stator motor connection 502 of arterial percutaneous endovascular centrifugal heart pump 300a is coupled with the motor junction 522 and motor 501. Hemodynamic support is started continuous flushing with special solution to maintain anticoagulation.

Arterial Percutaneous Endovascular Centrifugal Heart Pump Deployment into the Aortic

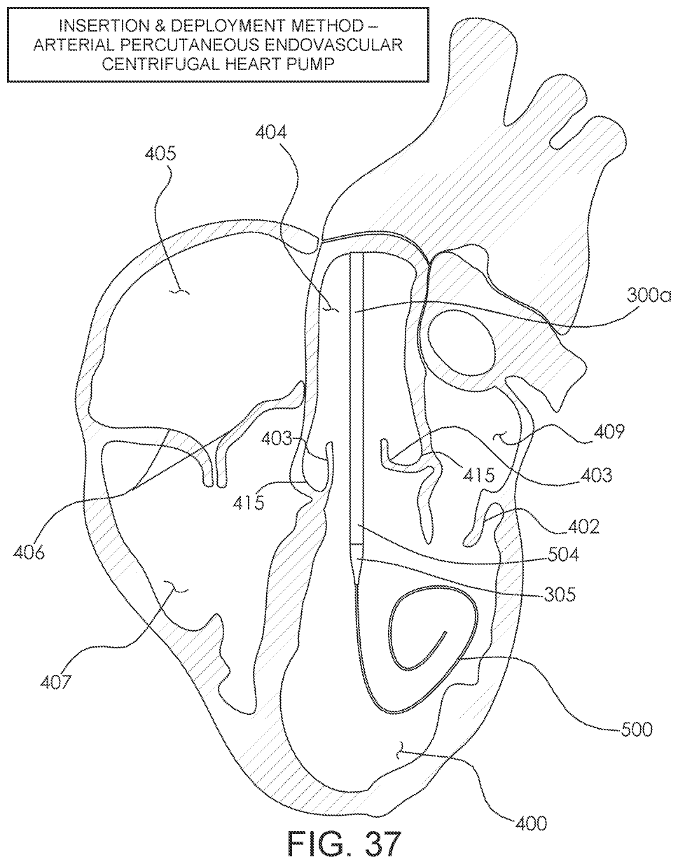

Referring to FIGS. 36-38
Left Ventricular wall 401
Mitral valve 402
Right atrium 405.
Tricuspid valve 406
Right ventricle 407
Right ventricular wall 408
Left atrium 409
Pulmonary valve leaflet tip 410
Pulmonary valve leaflet base 411
Pulmonary artery 412

The guidewire 500 is advanced across the ascending aorta 404 crossing the aortic valve leaflets 403 into the left ventricle 400. Once the guidewire 500 is in place, the arterial percutaneous endovascular centrifugal heart pump 300a is advanced along the guidewire 500 through the insertion tip inner lumen 309. When the Insertion tip 305 crosses the aortic valve leaflets 403, the unsheathing process may begin.

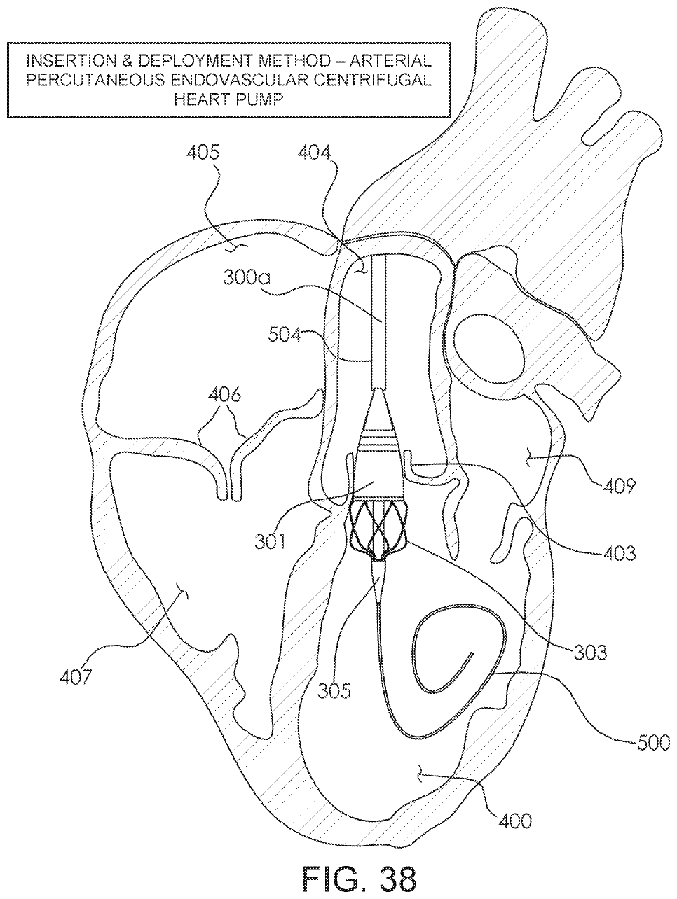

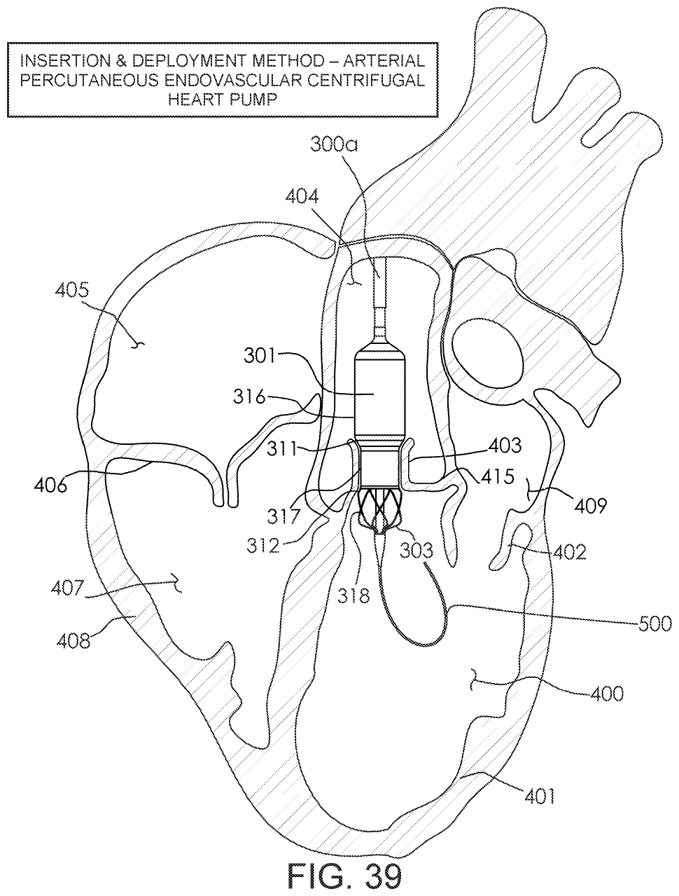

Referring now to FIGS. 38-40, when the arterial percutaneous endovascular centrifugal heart pump 300a is unsheathed—retracting the sheath 504 from the insertion to 305—the sheath is retracted. The frame 303 may begin to expand and interact with the aortic valve leaflets 403. When the arterial percutaneous endovascular centrifugal heart pump 300a is fully unsheathed the valve conduit 301 may fully interact with aortic valve leaflets 403. At this point, most of the valve conduit is in the ascending aorta 404. The frame midsection 317 may rest on the aortic valve leaflets 403, the bottom section of the valve anchored 312 may rest on the lower portion of the aortic valve leaflets 403, and the top section of the valve anchored 311 may also rest on the aortic valve leaflets 403 (FIG. 39).

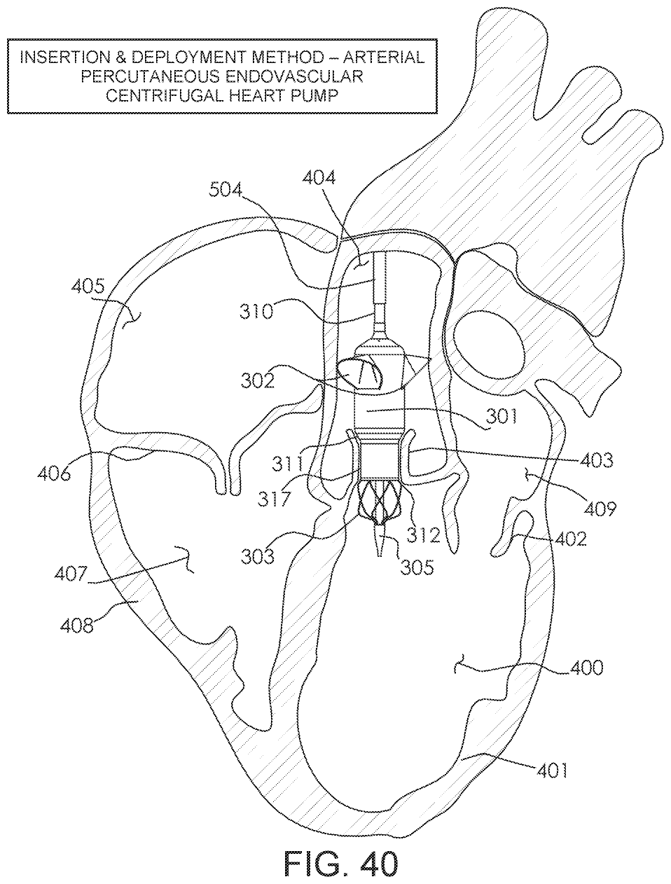

Once the arterial percutaneous endovascular centrifugal heart pump 300a is unsheathed, guidewire 500 is removed. The valve conduit valves 302 prevent blood flow from entering the left ventricle 400. As the left ventricle 400 contracts and generates positive pressure the valve conduit valves 302 open when the ventricular pressure is greater than the aortic pressure. Furthermore, if the aortic pressure is greater than the ventricular pressure then the valve conduit valves 302 close, thereby minimizing regurgitation flow. The valve conduit valves 302 allow time for the placement of the motor 501. Once the motor 501 is connected and turned on, the impeller 204 would generate pressure opening the valve conduit valves 302 and unloading the left ventricle 400 (See FIG. 40).

### Insertion of Venous Percutaneous Endovascular Centrifugal Heart Pump

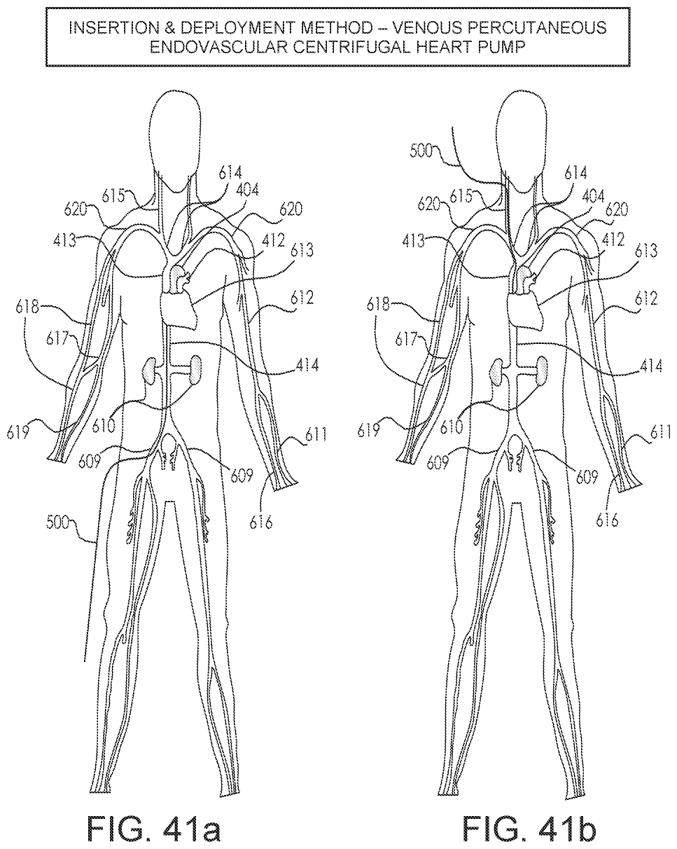

Referring to FIGS. 41a-b, 42a-b, 43
Femoral vein 609
Kidneys 610
Radial vein 611
Brachial vein 612
Right side heart 613
Brachiocephalic vein 614
Internal jugular vein 615
Ulnar vein 616
Medial cubital vein 617
Cephalic vein 618
Basilic vein 619

The insertion of the venous percutaneous endovascular centrifugal heart pump 300b has multiple points of entry in the human body 602.

The first point of entry is through the femoral vein 609. The guidewire 500 is inserted in the body 602 entering the femoral vein 609, it is advanced passing the kidneys 610 and inferior vena cava 414 (FIG. 41a). The guidewire 500 then enters the right side of the heart 613.

The second point of entry is through the jugular vein 615. The guide wire 500 is inserted into the body 602 entering the jugular vein 615, it is advanced passing the brachiocephalic vein and the superior vena cava 413 (FIG. 41b). The guidewire 500 then enters the right side of the heart 613.

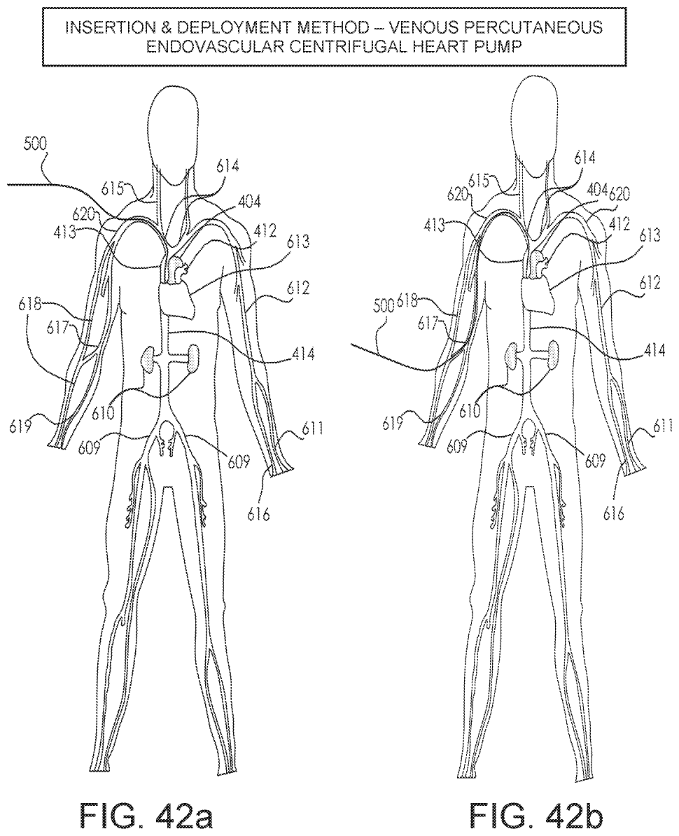

The third point of entry is through the subclavian vein 620. The guide wire 500 is inserted into the body 602 entering the subclavian vein 620, it is advanced passing the brachiocephalic vein and the superior vena cava 413 (FIG. 42a). The guidewire 500 then enters the right side of the heart 613.

The fourth point of entry is through the basilic vein 619, the medial cubital vein 617 or the cephalic vein 618. The guidewire 500 is inserted in the body entering the medial cubital vein 617, the basilic vein 619, or the cephalic vein 618. It is advanced passing the subclavian vein 620, the brachiocephalic vein 614, and the superior vena cava 413 (FIG. 42b). The guidewire 500 then enters the right side of the heart 613.

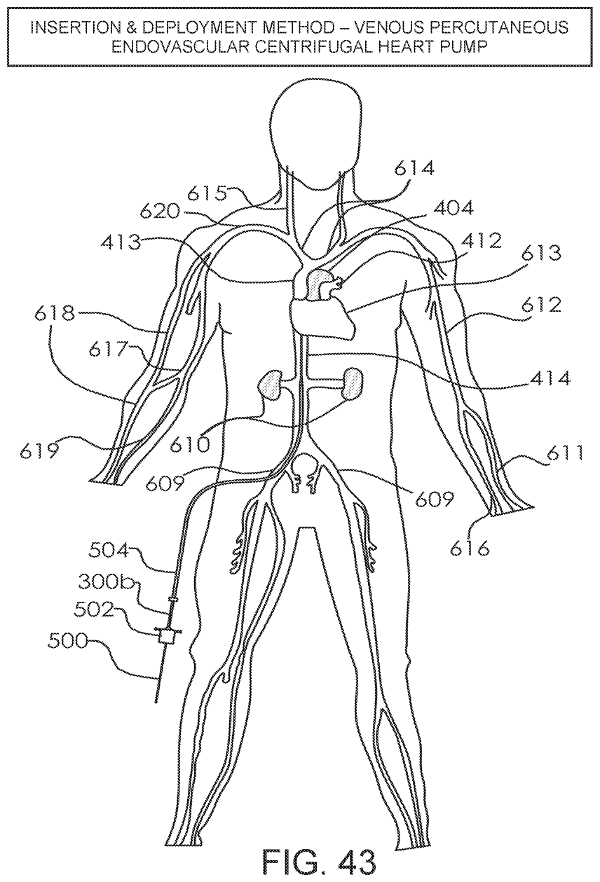

Guidewire 500 provides guidance for the venous percutaneous endovascular centrifugal heart pump 300b for each of these four points of entry (FIG. 43). The venous pump 300b has a hollow spacing throughout to allow the guidewire 500 to be inserted. As the guidewire 500 is inserted in the venous heart pump 300b, the venous heart pump 300b is advanced across the guidewire 500 into the body 602 following the path that the guidewire 500 has established. Throughout the insertion of the venous heart pump 300b, the guidewire 500 remain stationary. Thus, the venous heart pump 300b may enter the body through the four entry points noted: femoral vein 609, jugular vein 615, subclavian vein 620, and medial cubital vein 617 or cephalic vein 618.

Once the venous percutaneous endovascular centrifugal heart pump 300b has reached its final placement, it is unsheathed, followed by removal of the guidewire 500 and connection of the motor 501 (See FIG. 32). The motor connection for the venous percutaneous endovascular centrifugal heart pump 300b is the same as the arterial percutaneous endovascular centrifugal heart pump 300a.

Vascular access is obtained using anatomical landmarks, radiological landmarks and ultrasound guided vascular access. The objective is to access the femoral vein 609, jugular vein 615 or subclavian vein 620 in the anterior wall of the vessel, and veins of the upper extremeties.

The vein is accessed using the Seldinger technique, where a needle is advanced from the skin toward the vessel. When the needle is inside the vessel, a wire is advanced into the vein. The needle is withdrawn, and an insertion sheath is advanced over the wire into the selected vein. Once vascular access has been achieved the insertion sheath is suction and flushed. Anticoagulation is then started to achieved ACT levels between 250 to 300.

A pigtail catheter or a Swan Ganz catheter is advanced into the right atrium 405, right ventricle 407 and pulmonary artery 412. All pressures are recorded. The guidewire 500 (again preferably about 0.035 inches in diameter) is then advanced using the pigtail catheter or Swan Ganz catheter into the pulmonary artery 412 or its branches. Once the guidewire 500 is placed in the pulmonary artery 412 or one of its branches, the pigtail or the Swan Ganz catheter is removed, followed by the removal of the insertion sheath from the body 602. The venous percutaneous endovascular centrifugal heart pump 300b is then introduced and advanced into the pulmonary artery 412. When the collapsed valve conduit 301 segment of the sheath 504 is across the pulmonary valve, the sheath is retracted over the stator or the stator is advanced over the wire unsheathing the valve conduit 301 with the impeller inside. Once the valve conduit 301 is fully expanded across the pulmonary valve leaflet base 410 and pulmonary valve leaflet tip 411, the guidewire 500 is withdrawn from the body 602 and the stator motor connection 502 of venous percutaneous endovascular centrifugal heart pump 300b is coupled with the motor junction 522 and motor 501. Hemodynamic support is started continuously flushing with special solution to maintain anticoagulation.

### Pulmonary Placement of Venous Percutaneous Endovascular Centrifugal Heart Pump

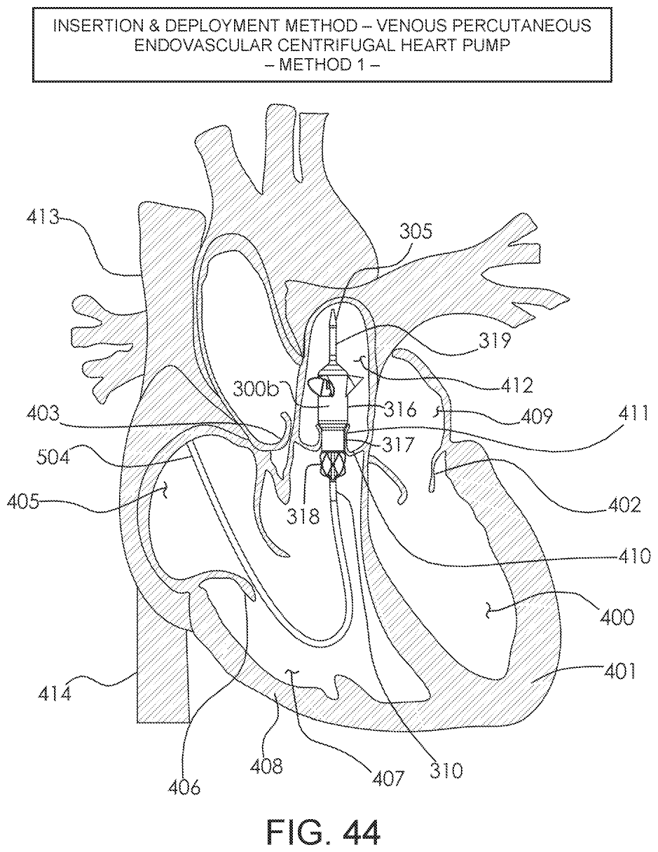

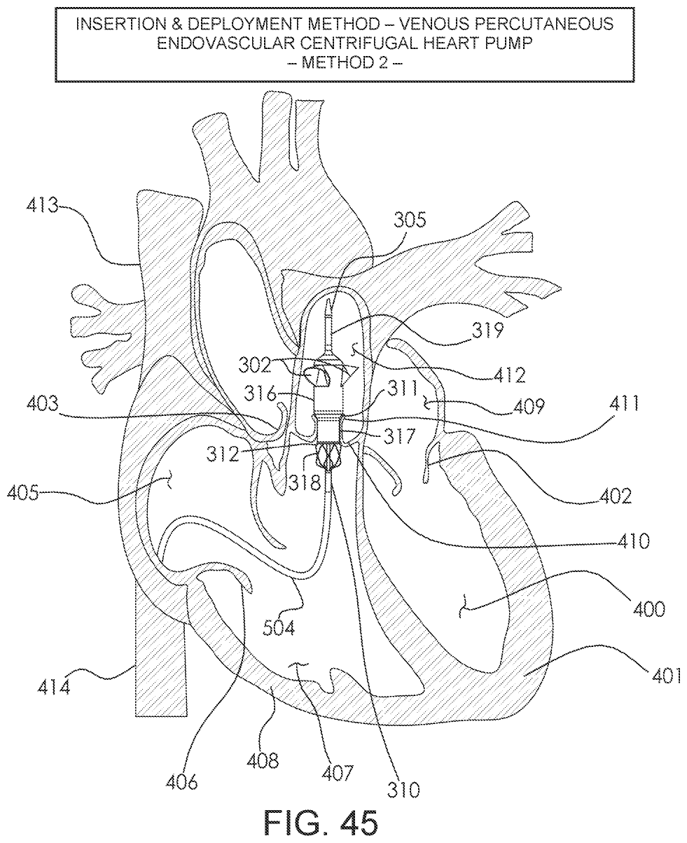

Referring to FIGS. 44-45
Pulmonary valve leaflet Tip 410
Pulmonary valve leaflet base 411
Pulmonary artery 412
Superior vena cava 413
Inferior vena cava 414

For pulmonary placement of the venous percutaneous endovascular centrifugal heart pump 300b, there are two methods of insertion.

Referring to FIG. 44, one method is advancing the guidewire 500 across the superior vena cava 413, crossing the tricuspid valve 406, entering the right ventricle 407, and crossing into the pulmonary artery 412. Furthermore, the venous percutaneous endovascular centrifugal heart pump 300b is advanced on the guidewire 500 in between the right ventricle 407 and pulmonary artery 412. Once this is achieved, the venous percutaneous endovascular centrifugal heart pump 300b is unsheathed by retracting the sheath 504 away from the insertion tip 305 (FIG. 44). During the unsheathing process, the valve conduit 301 contacts the pulmonary valve leaflet tip 411. Thus, the frame midsection 317 firmly anchors to the pulmonary valve leaflet tip 411 due to the enhanced diameter of the midsection 317 compared to the prior art. The frame lower section 318 remains in the right ventricle 407 and the frame top-section 316 remains in the pulmonary artery 412. Once deployed and delivery is satisfied, the guidewire 500 may be removed, followed by the connection of the motor. The advantage of the valve conduit valves 302a-c is that they replace the function of the native pulmonary valve minimizing regurgitant flow from occurring during the removal process of the guidewire 500 and connection of the motor 501. If the present invention is not placed satisfactory it may be recapture by repositioning.

Referring to FIG. 45, the second method is advancing the guidewire 500 across the inferior vena cava 414, crossing the tricuspid valve 406, entering the right ventricle 407, and crossing into the pulmonary artery 412. As mentioned previously, during the unsheathing process, valve conduit 301 contacts the pulmonary valve leaflet tip 411 while frame midsection 317 again firmly anchors itself to pulmonary valve leaflet tip 411. Frame lower section 318 remains in the right ventricle 407, and frame top-section 316 remains in the pulmonary artery 412. Once deployed and delivery is satisfied, the guidewire 500 is removed, followed by the connection of the motor. The advantage of the valve conduit valves 302a-c is that they replace the function of the native pulmonary valve minimizing regurgitant flow from occurring during the removal process of the guidewire 500 and connection of the motor 501.

### Tricuspid Placement of the Venous Percutaneous Endovascular Centrifugal Heart Pump

For tricuspid placement of the venous percutaneous endovascular centrifugal heart pump 300b, there are two methods of insertion.

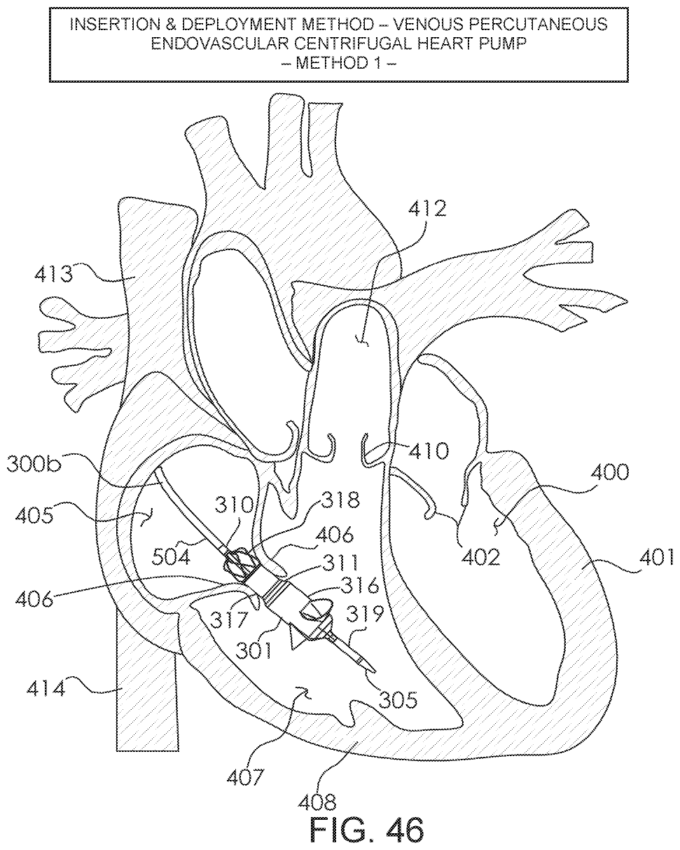

Referring to FIG. 46, one method is advancing guidewire 500 across the superior vena cava 413, crossing the tricuspid valve 406, and entering the right ventricle 407. The venous percutaneous endovascular centrifugal heart pump 300b is advanced over guidewire 500 between the right atrium 405 and right ventricle 407. Once this is achieved, the venous percutaneous endovascular centrifugal heart pump 300b is unsheathed by retracting the sheath 504 away from the insertion tip 305. During the unsheathing process, the valve conduit 301 contacts the tricuspid valve 406 and the frame midsection 317 anchors to tricuspid valve 406 due to the enhanced diameter of the midsection 317 compared to prior art devices. Frame lower section 318 remains in the right atrium 405 and frame top-section 316 remains in the right ventricle 407. Once deployed and delivery is satisfied, the guidewire 500 is removed, followed by connection of the motor. The advantage of the valve conduit valves 302a-c is that they replace the function of the native tricuspid valve 406 minimizing regurgitant flow from occurring during the removal process of the guidewire 500 and connection of the motor 501.

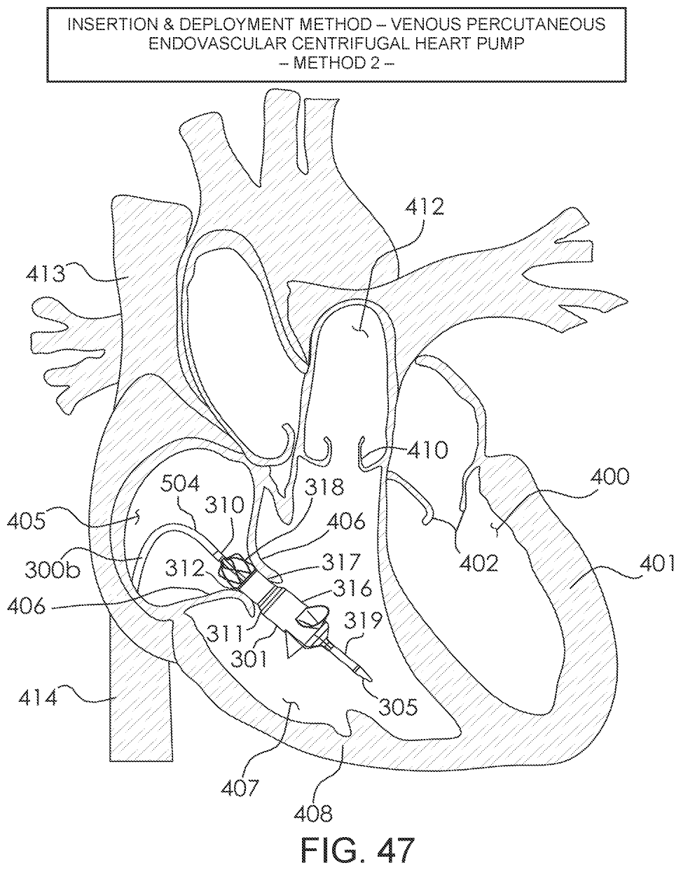

Referring to FIG. 47, the second method is advancing guidewire 500 across the inferior vena cava 414, crossing the tricuspid valve 406, and entering the right ventricle 407. The venous percutaneous endovascular centrifugal heart pump 300b is advanced over guidewire 500 between the right atrium 405 and right ventricle 407. Once this is achieved, the venous percutaneous endovascular centrifugal heart pump 300b is unsheathed by retracting sheath 504 away from the insertion tip 305. During the unsheathing process, the valve conduit 301 contacts the tricuspid valve 406 and the frame midsection 317 firmly anchors itself to the tricuspid valve 406 while frame lower section 318 remains in the right atrium 405 and frame top-section 316 remains in the right ventricle 407. Once deployed and delivery is satisfied, guidewire 500 is removed, followed by the connection of the motor. The advantage of the valve conduit valves 302a-c is that they replace the function of the native tricuspid valve 406 minimizing regurgitant flow from occurring during the removal process of the guidewire 500 and connection of the motor 501.

### Mitral Placement of the Venous Percutaneous Endovascular Centrifugal Heart Pump

For mitral placement of the venous percutaneous endovascular centrifugal heart pump 300b, there are two methods of insertion.

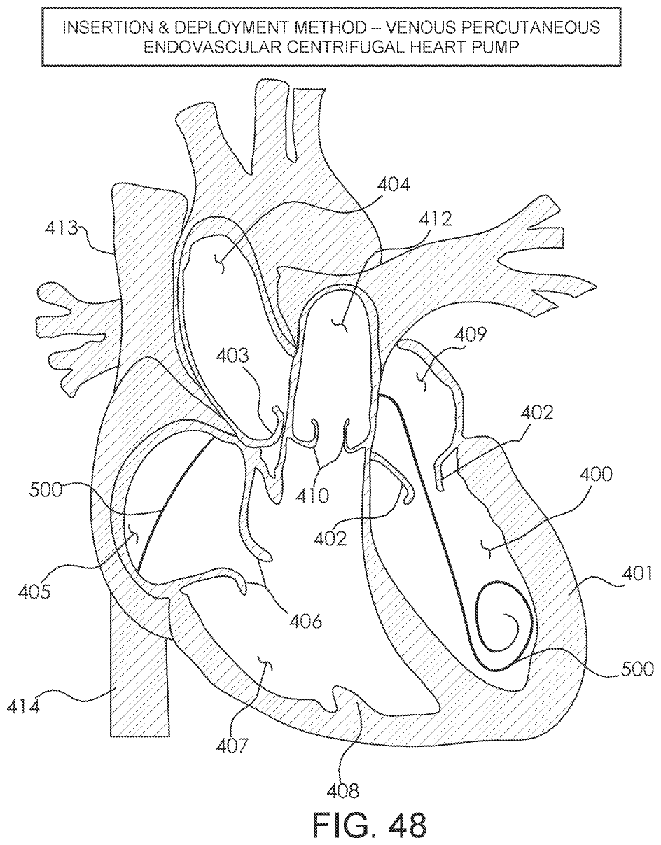

Referring to FIG. 48, one method is advancing guidewire 500 across the inferior vena cava 414, crossing the right atrium 405, crossing the atrial septum into the left atrium 409, and crossing the mitral valve 402 into the left ventricle 400. To achieve access to the left side of the heart, it is done by transseptal puncture, where a small puncture is made in the atrial septum located between the right atrium 405 and the left atrium 409. Thus, puncturing the right atrial 405 provides access to the left side of the heart. Furthermore, allowing the guidewire 500 to enter the left atrium 409 and crossing the mitral valve 402 into the left ventricle 400.

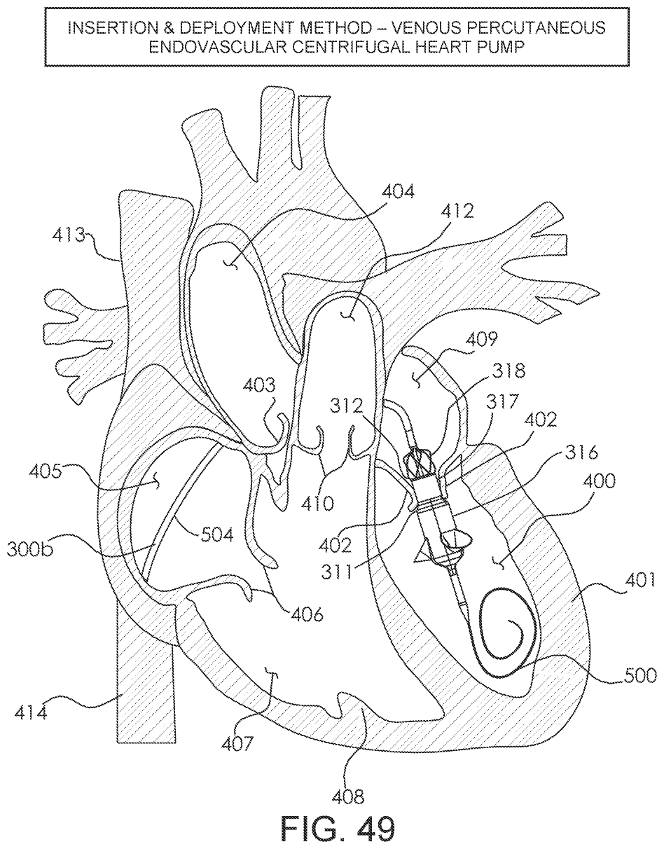

Referring to FIG. 49, once guidewire 500 is in the left ventricle 400, the venous percutaneous endovascular centrifugal heart pump 300b is advanced on the guidewire 500 crossing the right atrium 405 into the left atrium 409 and into the left ventricle 400. The venous percutaneous endovascular centrifugal heart pump 300b is unsheathed when it is in between the left atrium 409 and the left ventricle 400. Once this is achieved, the venous percutaneous endovascular centrifugal heart pump 300b is unsheathed by retracting the sheath 504 away from the insertion tip 305. The valve conduit 301 contacts mitral valve 402 and frame midsection 317 once again can firmly anchor itself to the mitral valve 402 while frame lower section 318 remains in the left atrium 409 and frame top-section 316 remains in the left ventricle 400. Once deployed and delivery is satisfied, the guidewire 500 is removed, followed by the connection of the motor. The advantage of the valve conduit valves 302a-c is that they replace the function of the native mitral valve 402 minimizing regurgitant flow from occurring during the removal process of the guidewire 500 and connection of the motor 501.

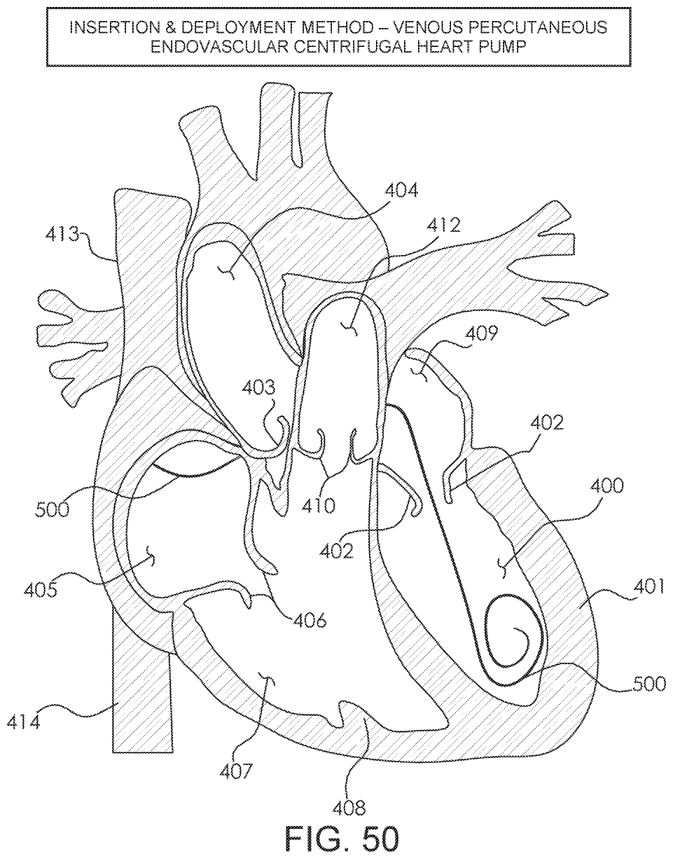

Referring to FIG. 50, the second method is by advancing guidewire 500 across the superior vena cava 413, crossing the right atrium 405 into the left atrium 409, and crossing the mitral valve 402 into the left ventricle 400. To achieve access to the left side of the heart, it is done by transseptal puncture, where a small puncture is made in the right atrium 405 that is connected to the left atrium 409. Thus, puncturing the right atrial 405 provides access to the left side of the heart allowing guidewire 500 to enter the left atrium 409 and crossing into the left ventricle 400.

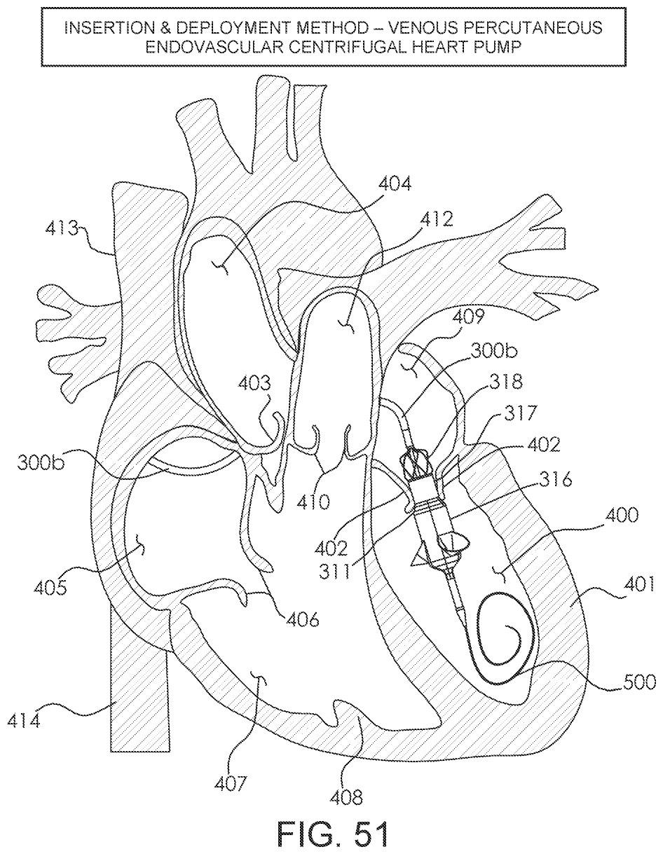

Referring now to FIG. 51, once guidewire 500 is in the left ventricle 400, the venous percutaneous endovascular centrifugal heart pump 300b is advanced on the guidewire 500 crossing the right atrium 405 into the left atrium 409 to the left ventricle 400. The venous percutaneous endovascular centrifugal heart pump 300b is unsheathed when it is in between the left atrium 409 and the left ventricle 400. The venous percutaneous endovascular centrifugal heart pump 300b is unsheathed by retracting the sheath 504 away from the insertion tip 305. The valve conduit 301 contacts the mitral valve 402 and the frame midsection 317 firmly anchors to the mitral valve 402 while frame lower section 318 remains in the left atrium 409 and frame top-section 316 remains in the left ventricle 400. Once deployed and delivery is satisfied, the guidewire 500 may be removed, followed by the connection of the motor. The advantage of the valve conduit valves 302a-c is that they replace the function of the native mitral valve 402 minimizing regurgitant flow from occurring during the removal process of the guidewire 500 and connection of the motor 501.

### Flow Profile

The percutaneous endovascular centrifugal heart pump 300 converts the mechanical energy of the fluids into hydraulic energy using centrifugal force. Impeller 204 uses centrifugal forces to expel the fluid radially converting axial flow to perpendicular flow. Impeller 204 suctions fluid in the same axis as valve conduit 301 and expels fluid perpendicular to the axis of valve conduit 301.

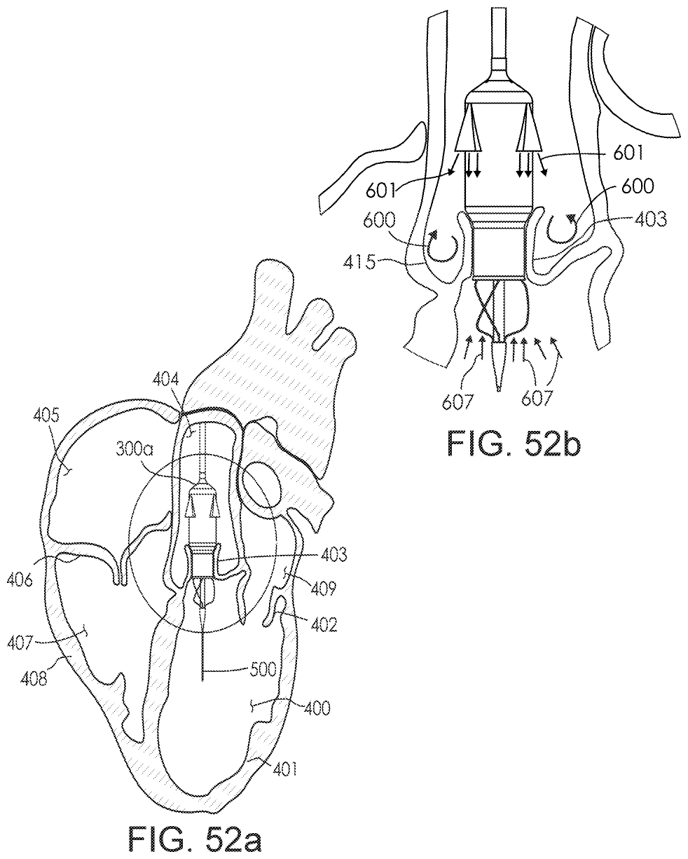

Referring to FIG. 52a-b, the percutaneous endovascular centrifugal heart pump 300 may use the valve conduit valves 302 to direct the flow depending on the angle of opening. As seen in FIG. 52b, when valve conduit valves 402 opening is limited, the outflow is directed downward 601 and lateral. This downward flow 601 is caused by valve conduit valves 402 limited opening directing the blood flow towards the coronary cusp 415. The flow directed downward may create recirculation flow 600 across the sinus of the aortic improving blood flow to the heart.

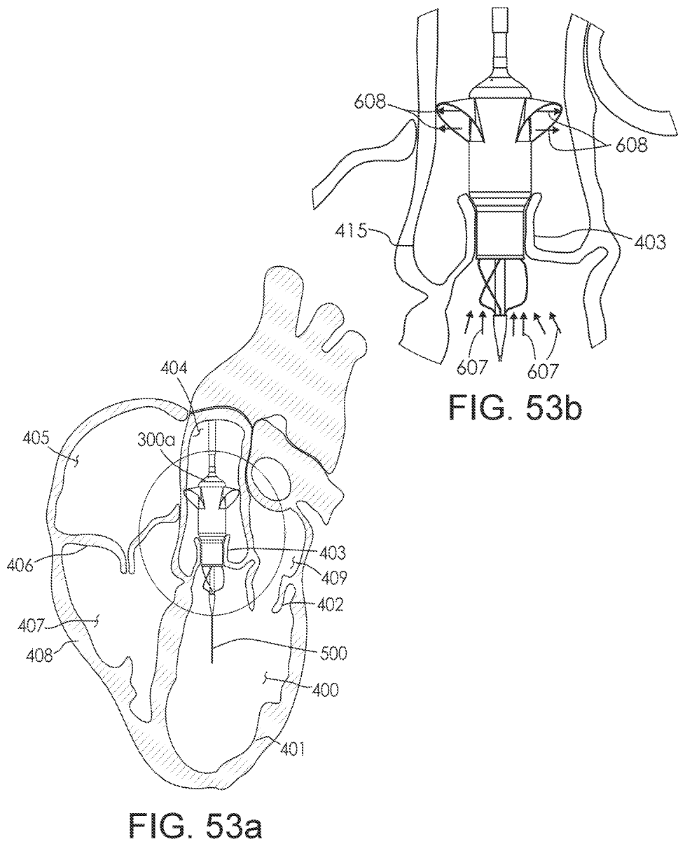

Referring now to FIGS. 53a-b, when the opening of the valve conduit valves 402 is increased, the outflow is expelled outwardly 608. This centrifugal flow is generated by impeller 204 design, which uses centrifugal forces to expel the fluid outwardly radially. FIG. 53b illustrates the flow direction created by the percutaneous endovascular centrifugal heart pump 300. The inflow 607 has an axial vector that is converted to a perpendicular vector at the outflow segment 608. This is achieved by means of impeller 204, which generates centrifugal forces.

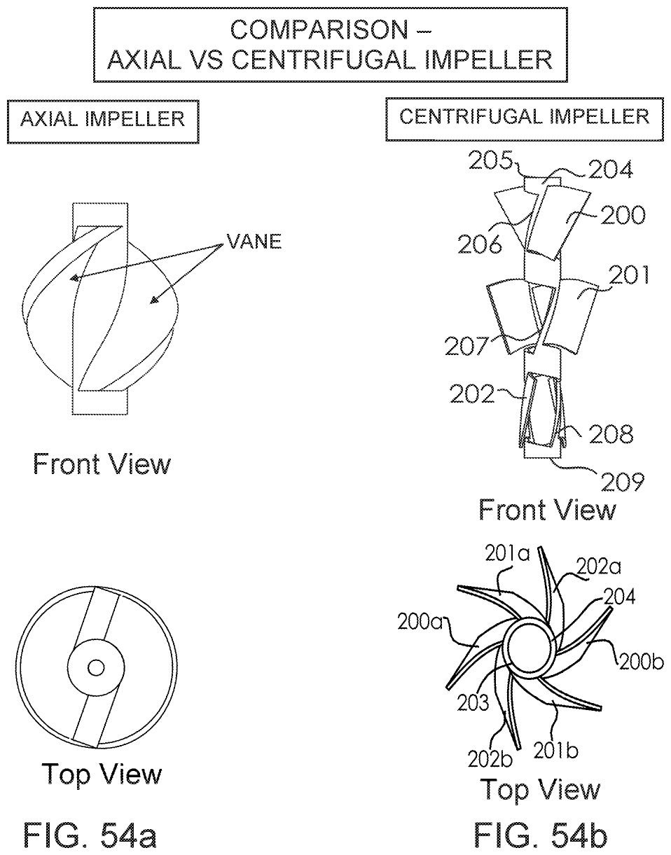

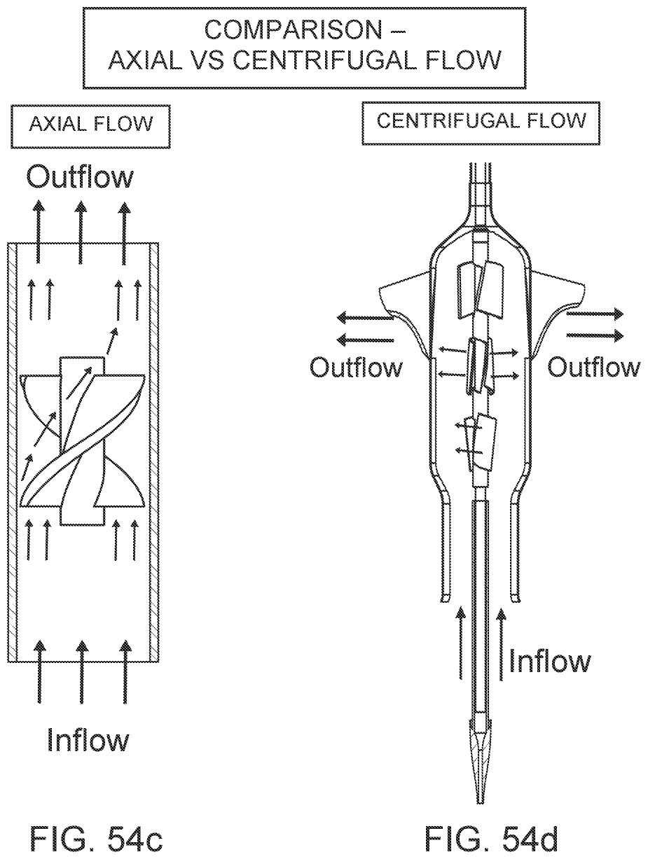

Referring to FIGS. 54a-b illustrate the differences in impeller design between an axial flow impeller and the percutaneous endovascular centrifugal heart pump impeller 204. In FIG. 54a, an axial flow impeller does not have any spacings and obstructs an axial view from the top view due to the vane design. However, referring to FIG. 54b, percutaneous endovascular centrifugal heart pump impeller has spacings between the top-level vane 200, mid-level vane 201, and lower-level vane 202; and the percutaneous endovascular centrifugal heart pump impeller does not obstruct an axial view from the top view of FIG. 54b.

FIGS. 54c and 54d further illustrates the differences in an axial impeller compared to percutaneous endovascular centrifugal heart pump impeller 204. An axial impeller couples the fluid to move the flow axially (FIG. 54c) in the same direction as the inflow. That is, axial pumps use a propeller to advance the fluid's mass on the same axis as the initial flow.

However, referring to FIG. 54d, the percutaneous endovascular centrifugal heart pump impeller 204 uses centrifugal forces to move the fluid perpendicular to the inflow, thus ejecting the fluid radially outwardly (perpendicular to the inflow). Centrifugal pumps generate flow by applying the angular momentum principle to the fluid's mass through the impeller passages advancing the mass of fluid radially.

### Electronics

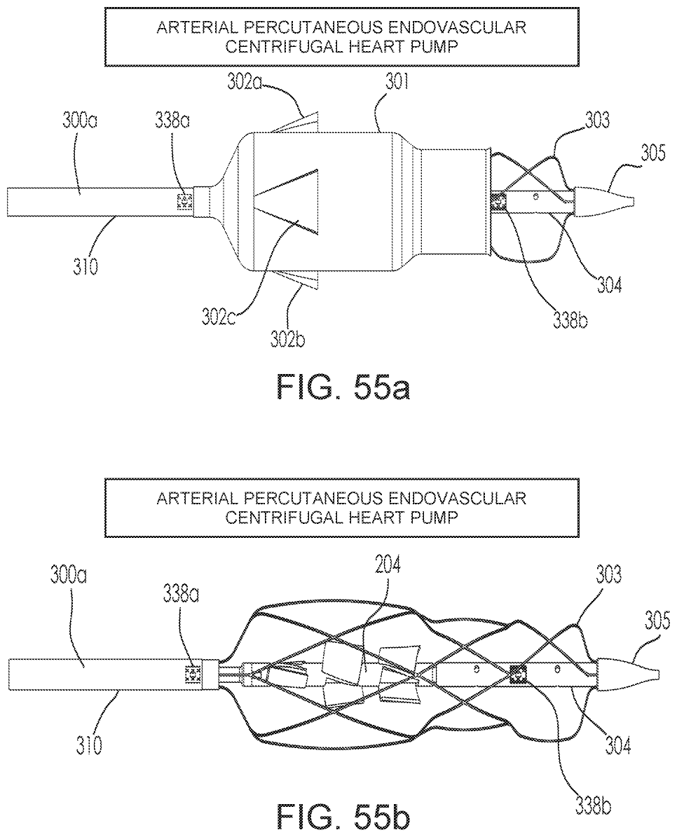

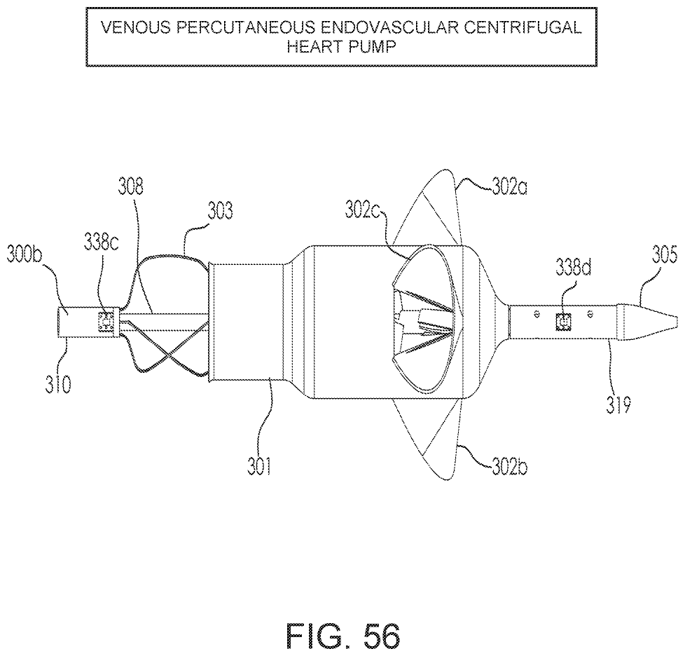

Referring now to FIGS. 55a-b and 56, the percutaneous endovascular centrifugal heart pump 300 may have sensors for the arterial percutaneous endovascular centrifugal heart pump 300a and venous percutaneous endovascular centrifugal heart pump 300b.

With reference to FIGS. 55a-b, the arterial percutaneous endovascular centrifugal heart pump 300a may include one or more microelectromechanical systems (MEMS) sensors in a proximal location 338a and a distal location 338b. Such sensors may measure pressure, temperature, position, flow, location, pH, lactate, etc. Proximal sensor 338a may be located on stator 310, and distal sensor 338b may be located on shaft stabilizer 304. The location of these sensors above and below the impeller permits the measurement of differential pressure across the device.

With reference to FIG. 56, the venous percutaneous endovascular centrifugal heart pump 300b may include one or more microelectromechanical systems (MEMS) sensors in a proximal location 338c and distal location 338d. Again, such sensors may measure pressure, temperature, position, flow, location, pH, lactate, etc. Proximal sensor 338c may be located on the stator 310, and distal sensor 338d may be located on the venous shaft stabilizer 319. Again, the location of these sensors above and below the impeller permits the measurement of differential pressure across the device.

Preferably, sensors 338a-d are powered by radiofrequency, and are commercially available such as model 1.2 BAR SCB10H-B012FB pressure sensor element from Murata Manufacturing Co., Ltd. of Nagaokakyo, Kyoto, Japan.

### Energy Transfer:

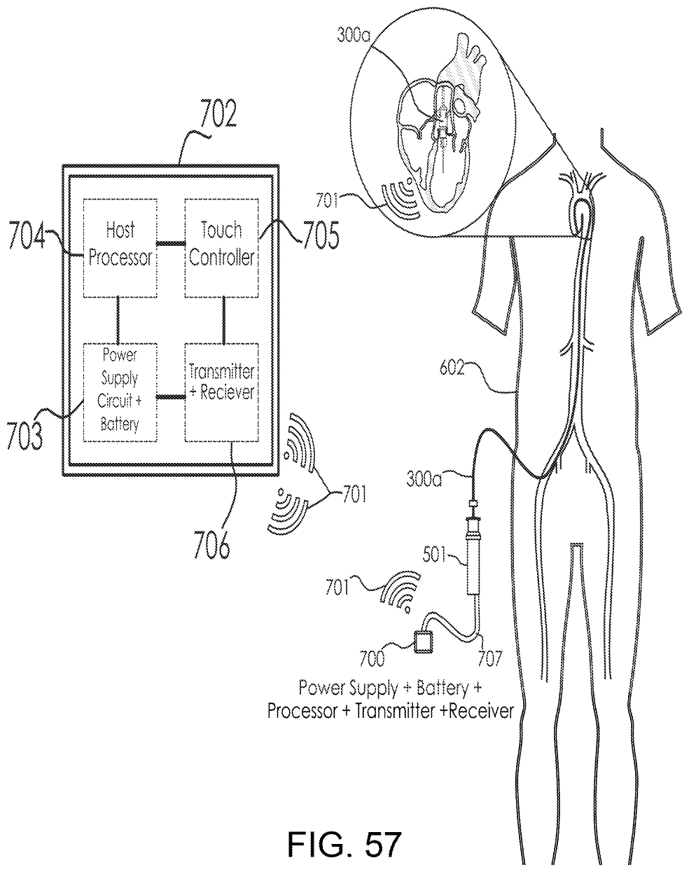

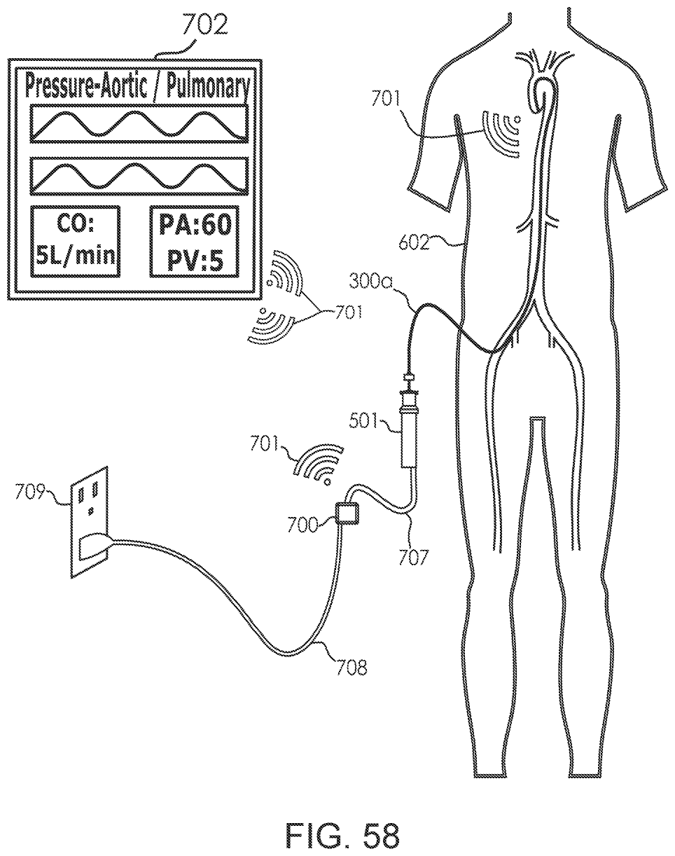

Referring now to FIGS. 57 and 58, the percutaneous endovascular centrifugal heart pump 300 may be connected to a power supply and processor 700 through motor cable 707. While FIGS. 57-58 shown the hookup for an arterial version of the present invention, the hookup for a venous version of the present invention would be the same.

The power supply and processor 700 may operate off a battery as shown in FIG. 57 or connected to an electrical outlet 709 by means of a power connector 708 as shown in FIG. 58. In either case, the power supply and processor 700 may communicate with a computer 702 by means of Bluetooth, radiofrequency, and/or Wi-Fi 701. The power supply and processor 700 may also communicate with sensors 338a-d of the percutaneous endovascular centrifugal heart pump 300 while in the patient's body. Thus, computer 702 may communicate as well with sensors 338a-d of the percutaneous endovascular centrifugal heart pump 300 in the patient's body.

Computer 702 may have a power supply circuit and battery 703, transmitter and receiver 706, host processor 704, and touch controller 705. The computer 702 may be a desktop computer such as Dell—Inspiron Compact Desktop (Dell Computer Company, Round Rock, TX), a laptop computer such as XPS 13 Laptop (Dell Computer Company, Round Rock, TX) or MacBook Pro (Apple, inc., Cupertino, CA), or a handheld smart phone such as an iPad or iPhone (Apple, inc., Cupertino, CA) or Samsung Galaxy Tablet or phone (Samsung Electronics Co., Ltd, Suwon-si, South Korea).

The computer 702 may thus generate a readout of the various parameters being received from sensors 338a-d, including operational values of the present invention such output rates, inflow rates, pressures, pH, temperature, impeller function and performance, and motor function and performance.

Having thus described in detail a preferred selection of embodiments of the present invention, it is to be appreciated and will be apparent to those skilled in the art that many physical changes could be made in the apparatus without altering the inventive concepts and principles embodied therein. The present embodiments are therefore to be considered in all respects as illustrative and not restrictive, the scope of the invention being indicated by the appended claims rather than by the foregoing description, and all changes which come within the meaning and range of equivalency of the claims are therefore to be embraced therein.

What is claimed is:

1. A percutaneous heart pump comprising:
an impeller having a proximal end and a distal end, and extendable blades rotatable outwardly creating centrifugal force;
a rotatable shaft attached proximate the proximal end of said impeller;
a non-rotatable stator supporting said shaft;
a valve conduit attached to said stator and having valves;
a non-rotatable expandable frame attachable to said valve conduit and circumscribing said impeller; and
a removeable sheath circumscribing said frame prior to said frame being expanded.

2. The percutaneous heart pump of claim 1, wherein said impeller comprises at least one level of at least two extendable vanes positioned equidistant along the circumference of said impeller.

3. The percutaneous heart pump of claim 2, wherein said impeller comprises at least two levels of extendable vanes, each level positioned along the longitudinal axis of said impeller and wherein said vanes at each level are offset circumferential from the vanes of adjacent levels according to the following equation:
$$α = \frac{180}{L}$$
where α is angular offset in degrees of adjacent levels of vanes and L is the number of levels.

4. The percutaneous heart pump of claim 1 further comprising a motor rotatably connected to said rotatable shaft for rotating said impeller.

5. The percutaneous heart pump of claim 1, wherein rotation of said impeller converts an axial flow along the longitudinal axis of said valve conduit into a transverse radially outwardly centrifugal flow through said valves.

6. The percutaneous heart pump of claim 5, wherein said impeller rotates between about 4000 revolutions per minute and about 25,000 revolutions per minute.

7. The percutaneous heart pump of claim 1, wherein prior to removal of said sheath said percutaneous heart pump comprises an outer diameter between about 1.5 mm and about 5 mm.

8. The percutaneous heart pump of claim 7, wherein the outer diameter of said percutaneous heart pump being preferably 3 mm.

9. The percutaneous heart pump of claim 1, wherein said frame being composed of a shapeable material comprises at least one anchor region adapted to contact the native leaflet of the heart.

10. The percutaneous heart pump of claim 9, wherein said frame comprises at least two anchor regions.

11. The percutaneous heart pump of claim 10, wherein said frame expands to a diameter between about 9 mm and about 20 mm.

12. The percutaneous heart pump of claim 11, wherein said frame expands to a diameter of about 15 mm.

13. The percutaneous heart pump of claim 1, wherein said valve conduit comprises at least three regions of varying diameter.

14. The percutaneous heart pump of claim 1, wherein said valves open and close as a function of pressure differential.

15. The percutaneous heart pump of claim 1, wherein said valves have a thickness between about 0.01 mm and about 0.30 mm.

16. The percutaneous heart pump of claim 15, wherein said valves have a thickness of about 0.05 mm.

17. The percutaneous heart pump of claim 1, wherein the percutaneous heart pump being for arterial application provides for commencement of axial flow proximate the distal end of the impeller along the longitudinal axis of said valve conduit and expulsion through the valves radially outwardly, and wherein the proximate end of said valve conduit being substantially sealed assists in the expulsion of flow through the valves radially outwardly.

18. The percutaneous heart pump of claim 17, wherein said sealed end includes at least one aperture.

19. The percutaneous heart pump of claim 18, wherein said aperture being between about 0.1 mm and about 3 mm, preferably about 0.5 mm.

20. The percutaneous heart pump of claim 1, wherein the percutaneous heart pump being for venous application provides for commencement of axial flow proximate the proximal end of the impeller along the longitudinal axis of said valve conduit and expulsion through the valves radially outwardly, and wherein the distal end of said valve conduit being substantially scaled assists in the expulsion of flow through the valves radially outwardly.

21. The percutaneous heart pump of claim 20, wherein said sealed end includes at least one aperture.

22. The percutaneous heart pump of claim 21, wherein said aperture being between about 0.1 mm and about 3 mm, preferably about 0.5 mm.

23. The percutaneous heart pump of claim 1 further comprising a shaft stabilizer attached to the distal end of the impeller.

24. The percutaneous heart pump of claim 23 further comprising at least one sensor affixed to said stator proximal said impeller.

25. The percutaneous heart pump of claim 24 further comprising at least two sensors, said second sensor affixed to said shaft stabilizer.

26. The percutaneous heart pump of claim 24 wherein said sensors measure pressure differential.

27. The percutaneous heart pump of claim 24 wherein at least one of said sensors measures temperature.

28. The percutaneous heart pump of claim 24 wherein at least one of said sensors measures direction and rate of fluid flow.

29. The percutaneous heart pump of claim 24 wherein at least one of said sensors measures pH.

30. The percutaneous heart pump of claim 24 wherein at least one of said sensors measures lactate.

31. The percutaneous heart pump of claim 1 wherein said impeller includes a drug capable of being eluted.

32. The percutaneous heart pump of claim 1 wherein frame includes a drug capable of being eluted.

33. A percutaneous heart pump comprising:
an impeller having a proximal end and a distal end, and extendable blades rotatable outwardly creating centrifugal force;
a rotatable shaft attached proximate the proximal end of said impeller, wherein said impeller comprises at least one level of at least two extendable vanes positioned equidistant along the circumference of said impeller;
a non-rotatable stator supporting said shaft;
a valve conduit attached to said stator and having valves radially displaceable as a function of pressure differential created by the rotation of said impeller.
a non-rotatable expandable frame being composed of a shapeable material comprising at least one anchor region adapted to contact the native leaflet of the heart; and
a sheath circumscribing said frame prior to said frame being expanded.

34. The percutaneous heart pump of claim 33 further comprising a motor rotatably connected to said rotatable shaft for rotating said impeller.

35. The percutaneous heart pump of claim 33, wherein said impeller rotates between about 4000 revolutions per minute and about 25,000 revolutions per minute.

36. The percutaneous heart pump of claim 33 wherein rotation of said impeller converts an axial flow along the longitudinal axis of said valve conduit into a transverse radially outwardly flow through said valves.

37. The percutaneous heart pump of claim 33, wherein said frame being composed of a shapeable material comprises at least one anchor region adapted to contact the native leaflet of the heart.

38. The percutaneous heart pump of claim 33, wherein said frame comprises at least two anchor regions.

39. The percutaneous heart pump of claim 33, wherein the percutaneous heart pump being for arterial application provides for commencement of axial flow proximate the distal end of the impeller along the longitudinal axis of said valve conduit and expulsion through the valves radially outwardly, and wherein the proximate end of said valve conduit being substantially sealed assists in the expulsion of flow through the valves radially outwardly.

40. The percutaneous heart pump of claim 39, wherein said sealed end includes at least one aperture.

41. The percutaneous heart pump of claim 40, wherein said aperture being between about 0.1 mm and about 3 mm, preferably about 0.5 mm.

42. The percutaneous heart pump of claim 33, wherein the percutaneous heart pump being for venous application provides for commencement of axial flow proximate the proximal end of the impeller along the longitudinal axis of said valve conduit and expulsion through the valves radially outwardly, and wherein the distal end of said valve conduit being substantially sealed assists in the expulsion of flow through the valves radially outwardly.

43. The percutaneous heart pump of claim 42, wherein said sealed end includes at least one aperture.

44. The percutaneous heart pump of claim 43, wherein said aperture being between about 0.1 mm and about 3 mm, preferably about 0.5 mm.

45. The percutaneous heart pump of claim 33 further comprising a shaft stabilizer attached to the distal end of the impeller.

46. The percutaneous heart pump of claim 33 further comprising at least one sensor affixed to said stator proximal said impeller.

47. The percutaneous heart pump of claim 46 further comprising at least two sensors, said second sensor affixed to said shaft stabilizer.

48. The percutaneous heart pump of claim 47 wherein said sensors measure pressure differential.

49. The percutaneous heart pump of claim 47 wherein at least one of said sensors measures temperature.

50. The percutaneous heart pump of claim 47 wherein at least one of said sensors measures direction and rate of fluid flow.

51. The percutaneous heart pump of claim 47 wherein at least one of said sensors measures pH.

52. The percutaneous heart pump of claim 47 wherein at least one of said sensors measures lactate.

53. The percutaneous heart pump of claim 33 wherein said impeller includes a drug capable of being eluted.

54. The percutaneous heart pump of claim 33 wherein frame includes a drug capable of being eluted.

55. A percutaneous heart pump comprising:
an impeller having a proximal end and a distal end, and extendable blades rotatable outwardly by centrifugal force;
a rotatable shaft attached proximate the proximal end of said impeller;
a non-rotatable stator supporting said shaft;
a valve conduit attached to said stator and having valves;
a non-rotatable expandable frame attachable to said valve conduit and circumscribing said impeller; and
a sheath circumscribing said frame prior to said frame being expanded, wherein said impeller comprises at least two levels of extendable vanes, each level positioned along the longitudinal axis of said impeller and wherein said vanes at each level are offset circumferential from the vanes of adjacent levels according to the following equation:
$$α = \frac{180}{L}$$
where α is angular offset in degrees of adjacent levels of vanes and L is the number of levels.

56. The percutaneous heart pump of claim 55, wherein the percutaneous heart pump being for arterial application provides for commencement of axial flow proximate the distal end of the impeller along the longitudinal axis of said valve conduit and expulsion through the valves radially outwardly, and wherein the proximate end of said valve conduit being substantially sealed assists in the expulsion of flow through the valves radially outwardly.

57. The percutaneous heart pump of claim 56, wherein said sealed end includes at least one aperture.

58. The percutaneous heart pump of claim 57, wherein said aperture being between about 0.1 mm and about 3 mm, preferably about 0.5 mm.

59. The percutaneous heart pump of claim 55, wherein the percutaneous heart pump being for venous application provides for commencement of axial flow proximate the proximal end of the impeller along the longitudinal axis of said valve conduit and expulsion through the valves radially outwardly, and wherein the distal end of said valve conduit being substantially sealed assists in the expulsion of flow through the valves radially outwardly.

60. The percutaneous heart pump of claim 55 wherein rotation of said impeller converts an axial flow along the longitudinal axis of said valve conduit into a transverse radially outwardly flow through said valves.

61. The percutaneous heart pump of claim 55, wherein said frame being composed of a shapeable material comprises at least anchor region adapted to contact the native leaflet of the heart.

62. The percutaneous heart pump of claim 60, wherein said frame expands to a diameter between about 9 mm and about 20 mm.

63. The percutaneous heart pump of claim 62, wherein said frame expands to a diameter of about 15 mm.

64. The percutaneous heart pump of claim 55, wherein said valve conduit comprises at least three regions of varying diameter.

65. The percutaneous heart pump of claim 60, wherein said impeller rotates between about 4000 revolutions per minute and about 25,000 revolutions per minute.

66. The percutaneous heart pump of claim 55, wherein prior to removal of said sheath said percutaneous heart pump comprises an outer diameter between about 1.5 mm and about 5 mm.

67. The percutaneous heart pump of claim 66, wherein the outer diameter of said percutaneous heart pump being preferably 2.5 mm.

68. The percutaneous heart pump of claim 55 further comprising a shaft stabilizer attached to the distal end of the impeller.

69. The percutaneous heart pump of claim 55 further comprising at least one sensor affixed to said stator proximal said impeller.

70. The percutaneous heart pump of claim 69 further comprising at least two sensors, said second sensor affixed to said shaft stabilizer.

71. The percutaneous heart pump of claim 70 wherein said sensors measure pressure differential.

72. The percutaneous heart pump of claim 70 wherein at least one of said sensors measures temperature.

73. The percutaneous heart pump of claim 70 wherein at least one of said sensors measures direction and rate of fluid flow.

74. The percutaneous heart pump of claim 70 wherein at least one of said sensors measures pH.

75. The percutaneous heart pump of claim 70 wherein at least one of said sensors measures lactate.

76. A method for installing a percutaneous heart pump in the human body comprising:
providing a heart pump having:
an impeller having a proximal end and a distal end, and extendable blades rotatable outwardly by centrifugal force,
a rotatable shaft attached proximate the proximal end of said impeller,
a non-rotatable stator supporting said shaft,
a valve conduit attached to said stator and having valves,
a non-rotatable expandable frame being manufactured of a shapable material attachable to said valve conduit and circumscribing said impeller, and
a removeable sheath circumscribing said frame prior to said frame being expanded;
routing the heart pump through a predetermined artery or vein into the human heart;
placing the valve conduit proximate a predetermined native valve of the human heart;
removing the sheath allowing the frame to expand to a predetermined shape;
anchoring the frame to contact a native leaflet of the predetermined valve; and
rotating the impeller causing an axial fluid flow transferred into a radial outwardly fluid flow through the valves.

77. The method according to claim 76 wherein the impeller is rotated between about 4000 revolutions per minute and about 25,000 revolutions per minute.

78. The method according to claim 76 wherein the heart pump further comprises at least two sensors supported proximate each end of the impeller.

79. The method according to claim 78 further comprising the step of measuring pressure differential.

80. The method according to claim 78 further comprising the step of measuring temperature.

81. The method according to claim 78 further comprising the step of measuring direction and rate of fluid.

82. The method according to claim 78 further comprising the step of measuring fluid pH.

83. The method according to claim 78 further comprising the step of measuring lactate.

84. The method according to claim 76 further comprising the step of ceasing rotation of the impeller and re-sheathing the frame prior to removal of the heart pump from the patient.

85. The method of claim 76 wherein the valve conduit is placed proximate the pulmonary valve.

86. The method of claim 76 wherein the valve conduit is placed proximate the tricuspid valve.

87. The method of claim 76 wherein the valve conduit is placed proximate the mitral valve.

88. A method for installing a percutaneous heart pump in the human body comprising:
providing a heart pump having:
an impeller having a proximal end and a distal end, and extendable blades rotatable outwardly by centrifugal force,
a rotatable shaft attached proximate the proximal end of said impeller,
a non-rotatable stator supporting said shaft,
a valve conduit attached to said stator and having valves,
a non-rotatable expandable frame being manufactured of a shapable material attachable to said valve conduit and circumscribing said impeller, and
a removeable sheath circumscribing said frame prior to said frame being expanded;
routing the heart pump through a predetermined artery or vein into the human heart;
placing the valve conduit proximate a predetermined native valve of the human heart;
removing the sheath allowing the frame to expand to a predetermined shape;
anchoring the frame to contact a native leaflet of the predetermined native valve;
re-positioning the heart pump to a preferred position;
re-anchoring the frame to contact a native leaflet of the predetermined native valve;
rotating the impeller causing an axial fluid flow transferred into a radial outwardly fluid flow through the valves.
re-sheathing the sheath; and
remove the heart pump.
